(19) United States
(12) Patent Application Publication
(10) Pub. No.: US 2016/0074351 A1
(43) Pub. Date: Mar. 17, 2016

(54) DISPENSING SYSTEM

(71) Applicants: HZNP Limited, Hamilton Pembroke (BM); Mallinkrodt, LLC, Hazelwood, MO (US)

(72) Inventors: Tejas Desai, Mississauga (CA); Ngoc Truc-Chi Vo, Longueil (CA); Brian Dean Doty, Saint Charles, MO (US); Ronald J. Brandel, Florissant, MO (US); Baohua Yue, Ballwin, MO (US)

(21) Appl. No.: 14/919,212

(22) Filed: Oct. 21, 2015

Related U.S. Application Data
(63) Continuation of application No. 13/657,711, filed on Oct. 22, 2012.

(51) Int. Cl. A61K 31/196

(52) U.S. Cl. CPC A61K 31/196

(57) ABSTRACT

The invention provides systems and methods for controlled dispensing of topical analgesics contained in a metered dispensing system. The systems and method are useful for treating signs and symptoms of osteoarthritis. The method includes depressing a hand pump to dispense a dose of a topical pain reliever in a viscous solution from the hand pump, wherein the hand pump is configured to dispense the dose within a tolerance specified by a corresponding label approved by a government regulatory agency; and spreading the topical solution on skin.

### CROSS-REFERENCES TO RELATED APPLICATIONS

This application is a continuation of and claims the benefit of U.S. patent application Ser. No. 13/657,711, filed Oct. 22, 2012, which is hereby incorporated by reference in its entirety for all purposes.

### FIELD OF THE INVENTION

This invention relates to systems and methods for delivering topical medications in a metered dose.

### BACKGROUND OF THE INVENTION

Topical analgesics are available in many dosage forms, including solutions, liquids, foams, gels, creams, ointments and the like. They are packaged in various container closure systems, such as tubes, bottles, pouches, and canisters. One common issue with many topical analgesic products is inaccurate unit dose dispensing from multi-dose containers, which may cause patient underdoses or overdoses.

A dosing card is a known method to measure topical semi-solid drugs. The commercially available Voltaren® antiphlogistics and antirheumatics in the form of gels is an example that uses a dosing card as the primary dose measurement method. When the medicine gel is squeezed out of a tube to cover a pre-specified area on the dosing card, it is assumed that the correct dose is dispensed. Although the specified area serves as a guide, it is not always easy for the users to cover the exact area every time, and its two dimensional nature also limits the dispensing accuracy.

What is needed in the art are new ways to deliver viscous transdermal medicines administered topically. Methods are needed that will allow accurate and convenient dosing of a viscous solution or gel for local topical analgesics. Methods for improving dosing accuracy are desired to prevent wrong dose quantities. The present invention satisfies these and other needs.

### BRIEF SUMMARY OF THE INVENTION

This present invention provides systems and methods for administering topical agents. As such, in one embodiment, the present invention provides: a method for administering a recommended dosage of a topical pain reliever in a viscous solution, the method comprising:

actuating a hand pump, the actuating:
a) causing a tappet having a rod connected thereto to compress a spring and the rod to slide with respect to a piston and enter a metering chamber holding a first predetermined amount of topical pain reliever in the viscous solution;
b) causing an outlet valve of the metering chamber to open, which allows the first predetermined amount of topical pain reliever in the viscous solution to flow into a channel or opening of the rod; and then
c) causing the piston to stroke when a limit stop of the tappet connects with a limit stop of the piston, wherein the first predetermined amount of topical pain reliever in the viscous solution is evacuated through an output duct of the tappet, which rises back up by action of the spring; and then
d) causing the outlet value of the metering chamber to close and create a vacuum inside the metering chamber and to fill the chamber;
e) optionally repeating steps a) through d) to deliver the recommended dose; and
f) spreading the viscous solution on skin.

In another embodiment, the present invention provides a method for treating the signs and symptoms of osteoarthritis, the method comprising:
depressing a hand pump to dispense 1/n of a dose of a topical pain reliever in a viscous solution from the hand pump, wherein n is a positive integer, wherein the hand pump is configured to dispense the 1/n of the dose within a tolerance specified by a corresponding label approved by a government regulatory agency; and spreading the topical solution on skin.

In yet another embodiment, the present invention provides a dispensing system for a topical pain reliever in a viscous solution, the system comprising:
a topical pain reliever in a viscous solution comprising sodium diclofenac;
a sealed bag holding the viscous solution; and
a hand pump in fluid communication with the viscous solution, the hand pump comprising:
   a pump housing defining a metering chamber;
   a tappet having a rod connected thereto to compress a spring and slide along a piston and enter a metering chamber holding a first predetermined amount of topical pain reliever in a viscous solution, wherein the metering chamber is sized for a throughput of 1/n dose of the sodium diclofenac, wherein n is a positive integer, wherein the first predetermined amount of topical pain reliever in the viscous solution is evacuated through an output duct of the tappet, which rises back up by action of the spring to dispense the 1/n of the dose within a tolerance specified by a corresponding label approved by a government regulatory agency.

In still yet another embodiment, the present invention provides a method for treating the signs and symptoms of osteoarthritis of the knee of a human patient with a topical diclofenac preparation, the method comprising:

dispensing a therapeutically effective amount of diclofenac from a topical diclofenac preparation packaged in a pharmaceutically acceptable hand pump;

applying the therapeutically effective amount of diclofenac to the knee,

wherein the patient attains therapeutic blood levels of diclofenac within 4 to 12 hours after administration of the preparation and maintains pain relief for about 12 hours after administration of the preparation.

Advantageously, the present invention improves patient compliance. In certain aspects, the methods and systems herein include patient instructions to promote proper use of the container and accurate dosing. These and other aspects, objects, and advantages will become more apparent when read with the detailed description and drawings which follow.

### BRIEF DESCRIPTION OF THE DRAWINGS

FIG. 1 illustrates a hand pump suitable for use in the present invention.

FIGS. 2A-2D illustrate the mean diclofenac concentrations with standard error bars after administration of PENNSAID in healthy volunteers (FIG. 2A) and methods of dispensing and applying PENNSAID (FIGS. 2B-2D).

FIGS. 3A-3B illustrate a run chart and a statistical graph summary, respectively, for the weight of a first conforming dose from each container (the next dose after the first product is discharged during priming).

FIGS. 4A-4B show capability analyses for unit dose weights at specification ranges of 0.8-1.2 g and 0.9-1.1 g, respectively.

FIG. 5 shows a statistical process control chart for unit dose weight.

### DETAILED DESCRIPTION

### I. Embodiments

The present invention provides methods of and systems for administering topical agents. In certain aspects, the methods and systems employ a non-pressurized system. In one embodiment, the present invention provides a method for administering a recommended dosage of a topical pain reliever in a viscous solution, the method comprising:
actuating a hand pump, the actuating:
a) causing a tappet having a rod connected thereto to compress a spring and the rod to slide with respect to a piston and enter a metering chamber holding a first predetermined amount of topical pain reliever in the viscous solution;
b) causing an outlet valve of the metering chamber to open, which allows the first predetermined amount of topical pain reliever in the viscous solution to flow into a channel or opening of the rod; and then
c) causing the piston to stroke when a limit stop of the tappet connects with a limit stop of the piston, wherein the first predetermined amount of topical pain reliever in the viscous solution is evacuated through an output duct of the tappet, which rises back up by action of the spring; and then
d) causing the outlet value of the metering chamber to close and create a vacuum inside the metering chamber and to fill the chamber;
e) optionally repeating steps a) through d) to deliver the recommended dose; and
f) spreading the viscous solution on skin.

In certain aspects, the topical pain reliever comprises sodium diclofenac. In certain aspects, the viscous solution or gel is about 1.5% to about 3% by weight of sodium diclofenac such as about 2% by weight of sodium diclofenac.

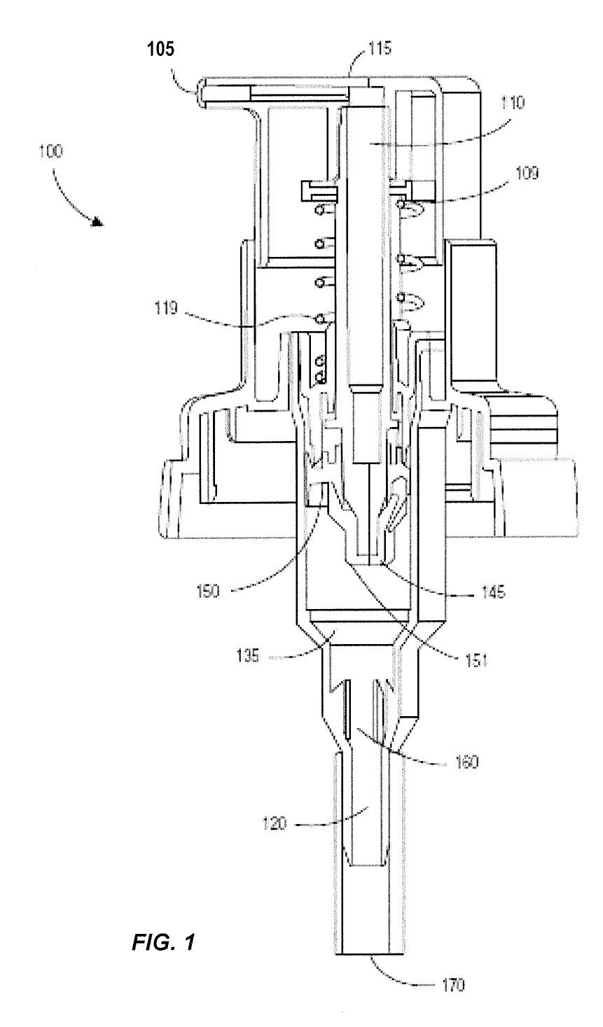

FIG. 1 is an illustration of a hand pump 100 suitable for use in the present invention. The system is available from Rexam PLC (of England and Wales) as a pump having SP943 as an identifier. A tappet 109 is activated by pressing the top 115 and compressing a spring 119. The tappet 109 is connected to a rod 110 having a channel 145, which slides along a piston 150 and enters a metering chamber 135. The metering chamber 135 is filled with a viscous solution, gel, or formulation. In operation, an outlet valve of the metering chamber opens, which allows the viscous solution, gel or formulation to flow into the channel or opening 145 from inlet 120. When a limit stop comes into contact with the piston 150, a venting hole allows the pressure inside the container to be the same as the atmospheric pressure. Then, with the piston 150 continuing its stroke, the viscous solution, gel or formulation is evacuated through the tappet's output duct. Once the product has been evacuated, the tappet is released and rises back up by action of the spring. The outlet valve closes in this movement, since the rod returns to a water-tight position. A vacuum is created inside the metering chamber, which makes a ball check valve 160 rise and open the way for the viscous solution, gel, or formulation to be sucked up into the dosage chamber. At the end of the piston rising stroke, the venting hole and the piston chamber are no longer in communication, which restores the overall water-tightness of the system.

The metering chamber 135 communicates with a dip tube 170 by means of a ball check valve 160. The piston 150 slides inside the pump body and, more particularly, inside the metering chamber 135. In one embodiment, a pump as disclosed in U.S. Pat. No. 7,243,820, issued Jul. 17, 2007, hereby incorporated by reference, is suitable for use in the present invention.

In certain aspects, the method further comprises aspirating a second predetermined amount of a topical pain reliever from a sealed bag holding the viscous solution, thereby shrinking the bag. In certain instances, pump 100 is disposed above the bag with dip tube 170 inserted in the bag. Preferably, the bag is metal lined or coated with polyester or polyethylene. Preferably, the metal pouch is inert to the pain reliever formulation. The metal can be made of aluminum or an aluminum alloy. In certain instances, the hand pump is elongated and the actuating occurs when the elongated hand pump is held at an angle with respect to a gravitational vector. In other instances, the hand pump is elongated and the actuating occurs when the elongated hand pump is upside-down with respect to a gravitational vector. An example of the liner is in the Rexam Sof'Bag, which is a foil laminate bag as an inner collapsible package.

In another embodiment, the present invention provides a method for treating signs and symptoms of osteoarthritis, the method comprising:
depressing a hand pump to dispense 1/n of a dose of a topical pain reliever in a viscous solution from the hand pump, wherein n is a positive integer, wherein the hand pump is configured to dispense the 1/n of the dose within a tolerance specified by a corresponding label approved by a government regulatory agency (e.g. United States Food and Drug Administration (FDA); and then spreading the topical solution on skin.

In certain instances, the dose tolerance is about ±0-20%, such as ±0, 1, 2, 3, 4, 5, 6, 7, 8, 9, 10, 11, 12, 13, 14, 15, 16, 17, 18, 19, or 20%. In other instances, the dose tolerance is about ±5-10%. The term “dose tolerance” includes the accuracy or precision of the first dose compared to subsequent dose, or as otherwise known in the art. For example, if the first dose is 1 g, a ±10% dose tolerance is between 0.9 to 1.1 g.

In certain instances, the integer “n” has a value from about 1 to about 10 such as 1, 2, 3, 4, 5, 6, 7, 8, 9 or 10. In one instance, n=1 and the depressing the hand pump a single time dispenses an entire dose of the viscous topical pain reliever in a viscous solution. In other instances, n=2, wherein the depressing of the hand pump twice dispenses a half-daily dose of the viscous topical pain reliever in a viscous solution. A daily dose may be two applications of 2 mL, or 4 actuations total.

In yet another embodiment, the present invention provides a dispensing system for a topical pain reliever in a viscous solution, the system comprising:
a topical pain reliever in a viscous solution comprising sodium diclofenac;
a sealed bag holding the viscous solution; and
a hand pump in fluid communication with the viscous solution, the hand pump comprising:
   a pump housing defining a metering chamber;
   a tappet having a rod connected thereto to compress a spring and slide along a piston and enter a metering chamber holding a first predetermined amount of topical pain reliever in a viscous solution, wherein the metering chamber is sized for a throughput of 1/n dose of the sodium diclofenac, wherein n is a positive integer, wherein the first predetermined amount of topical pain reliever in a viscous solution is evacuated through an output duct of the tappet, which rises back up by action of the spring to dispense the 1/n of the dose within a tolerance specified by a corresponding label approved by a government regulatory agency.

In certain preferred aspects, the sealed bag holding the viscous solution excludes air, thereby allowing the hand pump and sealed bag to dispense the viscous solution from any orientation with respect to a gravity vector. Preferably, the system further comprises a bottle, a can or other canister enclosing the bag.

A “gravity vector” includes a direction in which gravity exerts a force on matter or as otherwise known in the art.

### II. Topical Formulation

In certain aspects, the formulation or viscous solution of topical pain reliever is a non-steroidal anti-inflammatory (“NSAID”) drug or pharmaceutically acceptable salt thereof. More preferably, the non-steroidal anti-inflammatory is diclofenac, which can exist in a variety of salt forms, including sodium, potassium, or diethylamine forms. In a preferred embodiment, the sodium salt of diclofenac is used. Diclofenac sodium or sodium diclofenac can be present in a range of approximately about 0.1% to about 10%, such as about 1, 2, 3, 4, or 5% w/w. In a preferred aspect, the pump solution contains a viscous solution, or gel of is 2% w/w (mass/mass) diclofenac sodium.

“About” includes within a tolerance level of ±1%, ±2%, ±3%, ±4%, ±5%, ±10%, ±15%, ±20%, ±25%, or as otherwise known in the art.

In certain aspects, the present active ingredient formulation or viscous solution includes a penetration enhancer. The penetration enhancer can be dimethyl sulfoxide (“DMSO”) or derivatives thereof. The DMSO may be present in an amount by weight of about 1% to about 70%, and more preferably, between about 25% and about 60%, such as about 25, 30, 40, 45, 50, 55, or 60% w/w. Preferably, DMSO is used in the present invention at a concentration of about 40 to about 50% w/w, such as about 41, 42, 43, 44, 45, 46, 47, 48, 49 and 50% and all fractions in between such as about 44, 44.5, 45, 45.5, 46, 46.5%, and the like.

In certain aspects, the present invention includes a lower alkanol, such as methanol, ethanol, propanol, butanol or mixtures thereof. In certain embodiments, the alkanol is present at about 1 to about 50% w/w. Preferably, ethanol is used at about 1-50% w/w, such as 1, 5, 10, 15, 20, 25, 30, 35, 40, 45, or 50% w/w, and all fractions in between.

In certain aspects, the present invention includes a polyhydric alcohol, such as a glycol. Suitable glycols include ethylene glycol, propylene glycol, butylene glycol, dipropylene glycol, hexanetriol and a combination thereof. Preferably, propylene glycol is used at about 1-15% w/w, such as about 1, 2, 3, 4, 5, 6, 7, 8, 9, 10, 11, 12, 13, 14, or 15% w/w, and all fractions in between.

In certain embodiments, the present invention includes glycerol (also referred to herein as glycerin) at a concentration of 0-12% w/w. Preferably, glycerol is used at 1-4% w/w, such as about 1, 2, 3, or 4% w/w, and all fractions in between. In some embodiments, no glycerol is used in the formulation i.e., the viscous solution or gel optionally comprises glycerol.

In a preferred aspect, the topical pain reliever comprising a diclofenac solution has at least one thickening agent to make a viscous solution. The at least one thickening agent of the present invention may be an acrylic polymer (for example, Carbopol polymers, Noveon polycarbophils and Pemulen polymeric emulsifiers available commercially from Noveon Inc. of Cleveland, Ohio), an acrylic polymer derivative, a cellulose polymer, a cellulose polymer derivative, polyvinyl alcohol, poloxamers, polysaccharides or mixtures thereof. Preferably the at least one thickening agent is hydroxypropylcellulose (HPC) used such that the end viscosity is between about 10 and about 50000 centipoise (cps). More preferably the end viscosity is between about 500 and about 20000 cps or about 500-3000 cps or about 1000-2000 cps. In certain aspects, the thickening agent is present at about 1-10%, 1-5% or about 1-3% such as about 1, 2, 3, 4, 5, 6, 7, 8, 9 or 10% w/w and all numbers in between such as about 2.5% w/w.

The present viscous solution or gel formulation may optionally include at least one antioxidant and/or one chelating agent and/or preservative.

In addition, the topical formulations useful in the present invention can also comprise a pH adjusting agent. In one particular embodiment, the pH adjusting agent is a base. Suitable pH adjusting bases include bicarbonates, carbonates, and hydroxides such as alkali or alkaline earth metal hydroxide as well as transition metal hydroxides. Alternatively, the pH adjusting agent can also be an acid, an acid salt, or mixtures thereof. Further, the pH adjusting agent can also be a buffer. Suitable buffers include citrate/citric acid buffers, acetate/acetic acid buffers, phosphate/phosphoric acid buffers, formate/formic acid buffers, propionate/propionic acid buffers, lactate/lactic acid buffers, carbonate/carbonic acid buffers, ammonium/ammonia buffers, and the like. The pH adjusting agent is present in an amount sufficient to adjust the pH of the composition to between about pH 4.0 to about 10.0, more preferably about pH 6.9 to about 9.8. In certain embodiments, the unadjusted pH of the admixed components is between 8 and 10, such as 9, without the need for the addition of any pH adjusting agents.

In certain aspects, the pain reliever formulation or viscous solution comprising a non-steroidal anti-inflammatory drug (NSAID) has a viscosity of at least 100 cps. Preferably, the gel formulation has a viscosity of at least 500 cps. More preferably, the formulation has a viscosity of at least 1000 cps. In other embodiments, the viscosity is about 1000-10,000 cps, or about 10,000-15,000 cps, or about 15,000-20,000 cps. In a preferred embodiment, the viscous solution or formulations has a viscosity of about 800-5000 cps, such as about 800, 900, 1000, 2000, 3000, 4000, or 5000 cps and all numbers in between. For example, the formulation has a viscosity of about 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, or 2000 cps. Alternatively, the formulation has a viscosity of about 0-25 cps or 3-25 cps.

A “viscous solution” includes a solution having a viscosity greater than that of water (i.e., 1.0020 cps at 20° C.) or as otherwise known in the art.

In one embodiment, the topical pain reliever formulation comprises, consists essentially of, or consists of:
(i) diclofenac sodium;
(ii) DMSO;
(ii) ethanol;
(iii) propylene glycol;
(v) a thickening agent;
(vi) optionally glycerol; and
(vii) water.

In another embodiment, the formulation is used for treating signs and symptoms of osteoarthritis in a subject suffering from articular pain, by topical administration to an afflicted joint area of a subject a therapeutically effective amount of the formulation.

In another embodiment, the topical formulation comprises, consists essentially of, or consists of:
(i) about 1-2% w/w diclofenac sodium;
(ii) about 40-50% w/w DMSO;
(iii) about 23-29% w/w ethanol;
(iv) about 10-12% w/w propylene glycol;
(v) about 1-10% w/w thickener (such as hydroxypropyl cellulose); and
(vi) water to make 100% w/w.

In yet a further embodiment, the present invention provides a topical formulation comprising, consisting essentially of, or consisting of: a diclofenac sodium solution and at least one thickening agent, which can be selected from cellulose polymer, a carbomer polymer, a carbomer derivative, a cellulose derivative, polyvinyl alcohol, poloxamers, polysaccharides, and mixtures thereof.

In an aspect of the above embodiments, the thickening agents can be selected from cellulose polymers, carbomer polymers, a carbomer derivative, a cellulose derivative, polyvinyl alcohol, poloxamers, polysaccharides, and mixtures thereof.

In another aspect of the above embodiments, diclofenac sodium is present at about 2% w/w; DMSO is present at about 45.5% w/w; ethanol is present at about 23-29% w/w; propylene glycol is present at about 10-12% w/w; hydroxypropylcellulose (HY119) is present at about 0-6% w/w; glycerol is present at about 0-4%, and water is added to make 100% w/w. In other aspects, there is no glycerol in the gel formulation and hydroxypropylcellulose (HY119) is present at about 1-6% w/w. In further aspects, the end viscosity of the gel is about 500-5000 centipoise.

A feature of the above gel formulations is that when such formulations are applied to the skin, the drying rate is quicker and transdermal flux is higher than previously described compositions, such as those in U.S. Pat. Nos. 4,575,515 and 4,652,557. Additional features of the preferred formulations include decreased degradation of diclofenac sodium, which degrades by less than 0.04% over the course of 6 months and a pH of 6.0-10.0, for example around pH 9.0. In one aspect, the topical formulation is described in U.S. application Ser. No. 12/134,121, incorporated herein by reference.

In another embodiment, the topical formulation comprises, consists essentially of, or consists of:
(i) about 1-2% w/w diclofenac sodium;
(ii) about 40-50% w/w DMSO;
(iii) about 10-12% w/w propylene glycol;
(iv) about 1-30% w/w ethanol;
(v) optionally glycerine;
(vi) water; and
(vii) at least one thickening agent selected from the group consisting of a cellulose polymer, a carbomer polymer, a carbomer derivative, a cellulose derivative, hydroxypropyl cellulose and mixtures thereof.

Preferably the topical formulation has a viscosity of about 500-5000 cps, such as a viscosity of at least 1000 cps. In other embodiments, the viscosity is about 1000-10,000 cps, or about 10,000-15,000 cps, or about 15,000-20,000 cps. In a preferred embodiment, the viscous solution or formulations has a viscosity of about 800-5000 cps, such as about 800, 900, 1000, 2000, 3000, 4000, or 5000 cps and all numbers in between.

In one non-limiting example, the commercially available PENNSAID R 1.5% topical solution is packaged into a container with a metering pump. Instead of counting 40 drops for one dose with the current commercial container, one single actuation delivers the claimed unit dose of 1.2 mL.

In another non-limiting example, a viscous formulation, with 2% diclofenac sodium and a thickening agent, is filled into containers with metering pumps. One actuation accurately delivers 1 gram of the viscous formulation, which is half the claimed dose.

### III. Methods of Use

The topical pain reliever formulations are particularly suited for use in treating pain-related diseases, disorders, or conditions, such as the treatment of the signs and symptoms of osteoarthritis (OA) chronically. It is also useful for the treatment of other chronic joint diseases characterized by joint pain, degeneration of articular cartilage, impaired movement, and stiffness. Suitable joints include the knee, elbow, hand, wrist and hip. Preferably, the joint includes a knee.

Due to the properties of higher flux and in vivo absorption, it is believed that the formulations of the present invention can be administered at lower dosing than previously described formulations. In particular, the compositions of the invention can be used at twice a day dosing or once a day dosing in the treatment of OA. This is a significant improvement as lower dosing is associated with better patient compliance, an important factor in treating chronic conditions.

Suitable amounts per administration will generally depend on the size of the joint, which varies per individual and per joint, however a suitable amount may range from 0.5 μl/cm² to 4.0 μl/cm². Preferably the amount ranges from 2.0 to 3.0 μl/cm². In a preferred aspect, the dose is 1, 2, 3, or 4 times daily with each dose being between 0.1-4 mL. In a preferred aspect, the dose is 2 times a day at 2 mL per dose (a total of 4 mL/daily.)

### IV. Pharmacokinetic Properties

In vivo tools can demonstrate whether changes in a topical viscous solution affect local delivery. The FDA has identified four potential technologies to investigate:
(1) Pharmacokinetic studies may be used to demonstrate efficacy and/or to determine product performance of topical viscous solutions. Bioequivalence studies with pharmacokinetic or clinical endpoints may be conducted to demonstrate equivalence between two different topical viscous solutions. For many topical viscous solutions, the amount of drug reaching the systemic circulation can be detected in the plasma and compared between topical viscous solutions, although its relationship to local delivery is still unknown.
(2) Skin Stripping removes the top layers of the skin for assay of drug concentration.
(3) Microdialysis involves inserting a small semipermeable capillary tube into the dermis about 1000 mm under the skin. The capillary tube is permeable to the drug; therefore, as perfusion fluid flows through the capillary, it takes up drug from the extracellular fluid of the tissue.
(4) Near Infrared Spectroscopy detects a unique signal that indicates the concentration of a particular drug and offers the possibility of a noninvasive assay of drug delivery to the skin.

See Critical Path Opportunities for Generic Drugs, Office of Generic Drugs, Office of Pharmaceutical Science, Center for Drug Evaluation and Research, dated May 1, 2007 available on the FDA.gov website. A skin irritation and sensitization study may also assist in determining bioequivalence between topical viscous solutions, especially if the differences involve potential penetration enhancers. See FDA Draft Guidance on Diclofenac Sodium, Revised June 2011.

The viscous solution, a topical diclofenac preparation, disclosed herein comprises a therapeutically effective amount of diclofenac sodium such that the patient quickly attains sufficient plasma concentrations of diclofenac (e.g., within about 4-12 hours such as about 4, 5, 6, 7, 8, 9, 10, 11 or 12 hours) after administration of the solution and maintains pain relief for about 12 hours after administration of the solution. Although systemic circulation of diclofenac detected in plasma does not directly correlate to local delivery, the FDA, in some instances, relies on pharmacokinetic data to determine bioequivalence between two topical diclofenac solutions. If the 90% confidence intervals for the ratios of the geometric means (test: reference) of the AUC and Cₘₐₓ fall between 80% and 125%, the test is bioequivalent to the reference and is presumed to provide a similar therapeutic effect as the reference. See FDA Draft Guidance on Diclofenac Sodium, Revised June 2011.

The topical diclofenac preparation disclosed herein, when topically administered to a subject, may produce a plasma profile characterized by a mean Cₘₐₓ (peak plasma concentration) for diclofenac sodium from about 10.0 ng/mL to about 25.0 ng/mL. In another embodiment, the mean Cₘₐₓ for diclofenac sodium may range from about 12.4 ng/mL to about 19.5 ng/mL. In an additional embodiment, the mean Cₘₐₓ for diclofenac sodium may be about 12.5, 13.0, 13.5, 14.0, 14.5, 15.0, 15.1, 15.2, 15.3, 15.4, 15.5, 15.6, 15.7, 15.8, 15.9, 16.0, 16.5, 17.0, 17.5, 18.0, 18.5, 19.0 or 19.5 ng/mL. Moreover, the mean Cₘₐₓ for diclofenac sodium at steady state may range from about 12.0 ng/mL to about 30.0 ng/mL, or from about 15.6 ng/mL/mg to about 25.0 ng/mL/mg. In an additional embodiment, the mean Cₘₐₓ for diclofenac sodium at steady state may be about 15.5, 16.0, 16.5, 17.0, 17.5, 18.0, 18.5, 19.0, 19.5, 19.6, 19.7, 19.8, 19.9, 20.0, 20.5, 21.0, 21.5, 22.0, 22.5, 23.0, 23.5, 24.0, 24.5 or 25.0 ng/mL.

In an additional embodiment, the topical diclofenac preparation, when topically administered to a subject, may produce a plasma profile characterized by a mean AUC₀₋₁₂ for diclofenac sodium from about 60.0 ng·hr/mL to about 140.0 ng·hr/mL. In a further embodiment, the mean AUC₀₋₁₂ for diclofenac sodium may be from about 76.0 ng·hr/mL to about 124.0 ng·hr/mL. In another embodiment, the mean AUC₀₋₁₂ for diclofenac sodium may be about 75.0, 80.0, 85.0, 90.0, 91.0, 92.0, 93.0, 94.0, 94.5, 95.0, 95.5, 96.0, 96.5, 97.0, 97.5, 98.0, 99.0, 99.5, 100.0, 105.0, 110.0, 115.0, 120.0 or 125.0 ng·hr/mL.

In an additional embodiment, the topical diclofenac preparation, when topically administered to a subject, may produce a plasma profile characterized by a mean AUC₀₋₂₄ for diclofenac sodium from about 100 ng·hr/mL to about 300 ng·hr/mL. In a further embodiment, the mean AUC₀₋₂₄ for diclofenac sodium may be from about 150 ng·hr/mL/mg to about 250 ng·hr/mL. In another embodiment, the mean AUC₀₋₂₄ for diclofenac sodium may be about 150, 155, 160, 165, 170, 175, 180, 185, 190, 191, 192, 193, 194, 195, 196, 197, 198, 199, 200, 205, 210, 215, 220, 225, 240, 235, 240, 245 or 250 ng·hr/mL. Additionally, the mean AUC₀₋₂₄ for diclofenac sodium at steady state may range from about 200 ng·hr/mL to about 450 ng·hr/mL, or from about 250 ng·hr/mL to about 405 ng·hr/mL. In another embodiment, the mean AUC₀₋₂₄ for diclofenac sodium at steady state may be about 250, 260, 270, 280, 290, 300, 310, 311, 312, 313, 314, 315, 316, 317, 318, 319, 320, 321, 322, 323, 324, 325, 330, 340, 350, 360, 370, 380, 390, 400 or 405 ng·hr/mL.

In a further embodiment, the topical diclofenac preparation, when topically administered to a subject, may produce a plasma profile characterized by a median Tₘₐₓ (time to peak plasma concentration) for diclofenac sodium from about 4.0 hours to about 12.0 hours. In an alternate embodiment, the median Tₘₐₓ for diclofenac sodium may be from about 6.0 hours to about 10.0 hours. In another embodiment, the median Tₘₐₓ for diclofenac sodium may be about 4.0, 4.5, 5.0, 5.5, 6.0, 6.5, 7.0, 7.5, 8.0, 8.5, 9.0, 9.5, 10.0, 10.5, 11.0, 11.5 or 12.0 hours. Moreover, the median Tₘₐₓ for diclofenac sodium at steady state may range from about 0 hours to about 12.0 hours, or from about 0.5 hours to about 8.0 hours relative to the time of administration of the last dose. In another embodiment, the median Tₘₐₓ for diclofenac sodium at steady state may be about 0.5, 1.0, 1.5, 2.0, 2.5, 3.0, 3.5, 4.0, 4.5, 5.0, 5.5, 6.0, 6.5, 7.0, 7.5 or 8.0 hours.

In certain aspects, the methods and systems of the present invention provide upon application of the topical diclofenac preparation in a viscous solution to the knee of a patient an AUC₀₋₂₄ for diclofenac of about 195.51±166.03 ng·h/mL.

In certain aspects, the methods and systems of the present invention provide upon application of the topical diclofenac preparation in a viscous solution to the knee of a patient a Cₘₐₓ for diclofenac of about 15.57±12.96 ng/mL.

In certain aspects, the methods and systems of the present invention provide upon application of the topical diclofenac preparation in a viscous solution to the knee of a patient an AUC₀₋₂₄ at steady state for diclofenac of about 319.51±162.36 ng·h/mL.

In certain aspects, the methods and systems of the present invention provide upon application of the topical diclofenac preparation in a viscous solution to the knee of a patient a Cₘₐₓ at steady state for diclofenac of about 19.79±10.12 ng/mL.

In certain aspects, the methods and systems of the present invention provide upon application of the topical diclofenac preparation in a viscous solution to the knee of a patient a mean AUC₀₋₁₂ for diclofenac of about 76.0 ng·hr/mL to about 124.0 ng·hr/mL.

In certain aspects, the topical diclofenac preparation described herein achieves the plasma profiles disclosed herein when administered from hand pump 100 (FIG. 1). In one aspect, the hand pump delivers 0.5-5 mL per pump action, such as 1, 2, 3, 4, or 5 mL per pump action. In a preferred aspect, the pump action is 1 mL per pump.

In certain aspects, the present invention provides a method for treating the signs and symptoms of osteoarthritis of the knee of a human patient with a topical diclofenac preparation, the method comprising:
dispensing a therapeutically effective amount of diclofenac from the topical diclofenac preparation packaged in a pharmaceutically acceptable hand pump;
applying the therapeutically effective amount of diclofenac to the knee; and, wherein the patient attains therapeutic blood levels of diclofenac within 4 to 12 hours such as 4, 5, 6, 7, 8, 9, 10, 11 or 12 and all fractions in between after administration of the preparation and maintains pain relief for about 12 hours after administration of the preparation.

In one aspect, patients are given the following instructions. 1) Remove the pump cap. 2) To prime the pump, while holding the bottle in an upright but tilted position, press the pump down firmly and fully several times into a paper towel or tissue until some study drug comes out. 3) After priming the pump, the bottle is ready to use and does not need to be primed again. 4) To dispense the drug, press the pump head down firmly and fully to dispense the drug into the palm of the hand. Release the pump head. 5) Apply the drug evenly over the entire application area.

### V. Examples

### Example 1

### Illustrates the Performance Evaluation of a 100 mL Rexam Sof'Bag Bottle with 1 mL Dispensing Pump for Diclofenac Sodium Topical 2.0% w/w

1. Summary

The current study was performed to evaluate the dose delivery attributes of the Rexam Sof'Bag container with a metering pump for diclofenac sodium topical gel 2.0% w/w. The study was conducted on 16 containers over a one month (16 business days) period. Results show that the container, when filled to full capacity, is capable of consistently delivering the required number of doses with high precision. The mean unit dose weight delivered from each container varied from 0.97 to 0.99 g, which is well within 5% of the target dose (1.00 g). Unit dose weight standard deviations from each bottle were stable at 0.01 g.

2. Introduction

Diclofenac sodium topical gel 2.0% w/w is a drug under investigation for the treatment of the signs and symptoms of osteoarthritis of the knee(s).

According to FDA guidance document “Container Closure Systems for Packaging Human Drugs and Biologics,” container performance includes “functionality and/or drug delivery” studies. This study was conducted to evaluate the following performance aspects: container priming parameters, delivery accuracy and precision, number of conforming doses, percentage of restitution, residual volume, and waste volume.

3. Materials, Equipment, Methods and Procedures

The study was conducted with a Rexam bottle and pump head. The inner pouch of the Rexam 100 mL bottle has a pressure rating of a half bar (7.2 psi). All instruments, including pressure gauge and balance were within calibration during this study.

Delivery attributes were studied over a total of sixteen (16) business days. The containers were filled, pumps were primed, and the first two dispensing sessions were performed. Containers were stored at room temperature during this period.

4. Results and Discussion

4.1. Container Fill Weight and Deliverable

Bottles were manually inflated at 6 psi prior to filling. Each container was filled to full capacity: just below the bottom of bottle neck and top of the shoulder. The container weights before and after filling, and before and after dispensing were recorded. Container fill capacity and deliverable weights were calculated. The total collected sample weight from each container was also calculated by summation of individual unit dose weights. Key statistics are summarized below in Table 1.

TABLE 1
Overall container fill capacity and deliverable

| | Empty | Initial | Total | End | | | | | |
|---|---|---|---|---|---|---|---|---|---|
| | container | gross | fill | gross | | Total | Total | | |
| | weight | weight | weight | weight | Residual¹ | delivered | collected | Restitution | Yield |
| | (g) | (g) | (g) | (g) | (g) | (g) | (g) | (%)² | (%) |
| Mean | 45.74 | 179.79 | 134.06 | 50.19 | 4.46 | 129.60 | 128.81 | 96.08 | 99.39 |
| STDEV | 0.04 | 1.20 | 1.21 | 0.75 | 0.74 | 1.58 | 1.68 | 0.59 | 0.29 |
| Minimum | 45.67 | 177.31 | 131.57 | 49.09 | 3.34 | 126.51 | 125.69 | 94.94 | 98.51 |
| Maximum | 45.84 | 181.75 | 136.08 | 51.62 | 5.88 | 132.34 | 131.51 | 96.93 | 100.09 |

¹Residual: Product left in bottle after completion of dispensing.
²Restitution: Percentage of total collected in total fill weight.
3 Yield: Percentage of total collected in total delivered.

Under the filling conditions used in this study, the deliverable weight (total delivered) results meet the requirements of USP <698> for multiple-unit containers. The average deliverable weight is NLT 100% of 120 g minimum requirement, and no individual is less than 95% of minimum.

4.2. Priming Requirement

The initial data collected at the beginning of bottle dispensing was used to assess the number of actuations required to fully prime the pump. Data indicated that once product is discharged, the next actuation always results in a full dose.

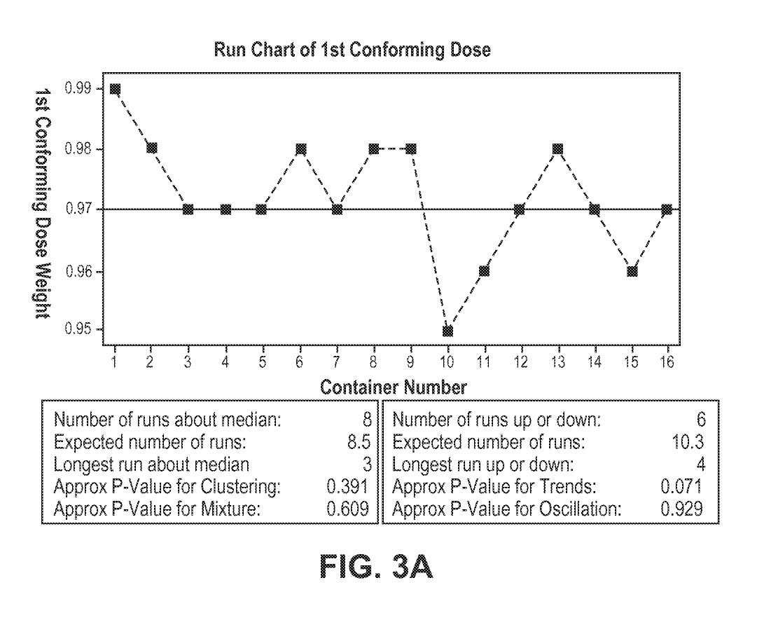

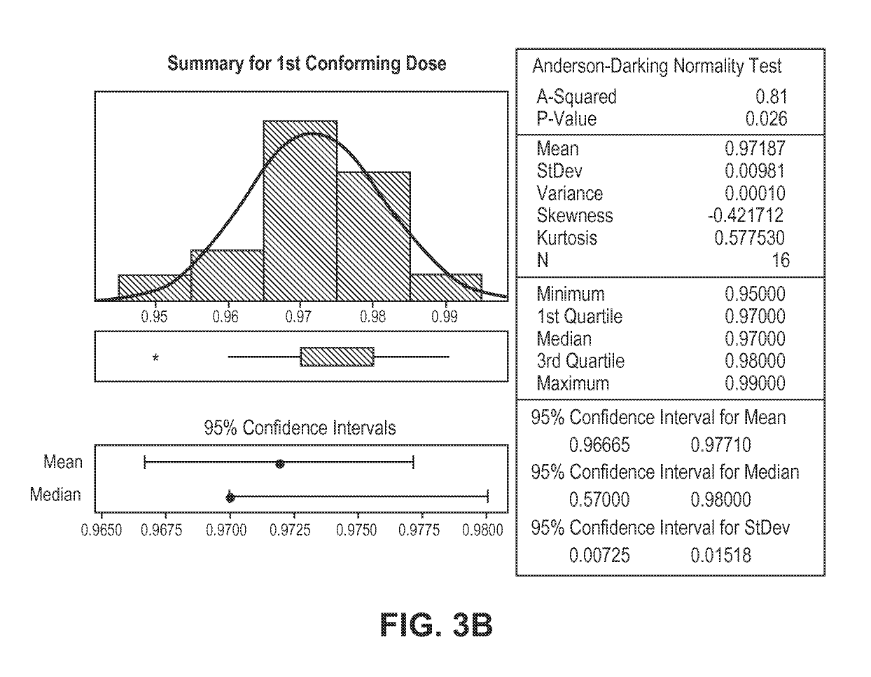

FIGS. 3A-3B show a run chart (FIG. 3A) and a statistical summary of the first conforming dose from all containers (FIG. 3B).

The number of priming actuations depends on container fill level. In this study, all containers were filled to full capacity, and two, three, four, or five actuations were required to fully prime the pump, depending on how full the bottles are filled.

Study results demonstrated that each bottle was capable of delivering more than 120 conforming doses. For consistency and comparison across all containers, only the first 120 consecutive doses after priming are analyzed in this session. Conforming doses during priming and at the end are described in 4.4.

4.3.1. Overall Statistics

Overall statistics are listed in Table 2 below. Across all 16 containers, dose number 33 was consistently the lowest dose among the 120 unit doses. This was caused by solvent evaporation through the pump head after standing for two weeks.

TABLE 2
Statistical summary of individual dose weight from all containers

| Statistics | Weight (g) |
|---|---|
| Individual Container - Maximum Mean | 0.99 |
| Individual Container - Minimum Mean | 0.97 |
| All Containers - Mean | 0.98 |
| All Containers - Standard Deviation | 0.01 |
| Maximum Single Actuation | 1.00 |
| Minimum Single Actuation (excluding dose | 0.93 |
| No. 33, 1st dose after long gap period.)** | |
| Minimum Single Actuation (dose No. 33) | 0.86 |

*Calculations were based on the first 120 consecutive doses after priming sessions.
**Unit dose No. 33 was dispensed on January 3rd, 2011, two weeks after the previous dose was dispensed.

4.3.2. Capability Analysis

The current proposed product specification requires the average dose weight to be within 0.8-1.2 g. As shown in Table 2 above, mean unit dose weights are all well within specification. To further examine container pump performance, a capability analysis was performed on all unit doses sub-grouped by containers. Results demonstrated a high capability to consistently deliver unit doses in the 0.8-1.2 g range. Even when the range is set between 0.9-1.1 g, an overall Cpk of 2.89 and Ppk of 1.86 were obtained. The capability numbers will be higher if a special event, i.e. dose number 33, is excluded from the analysis.

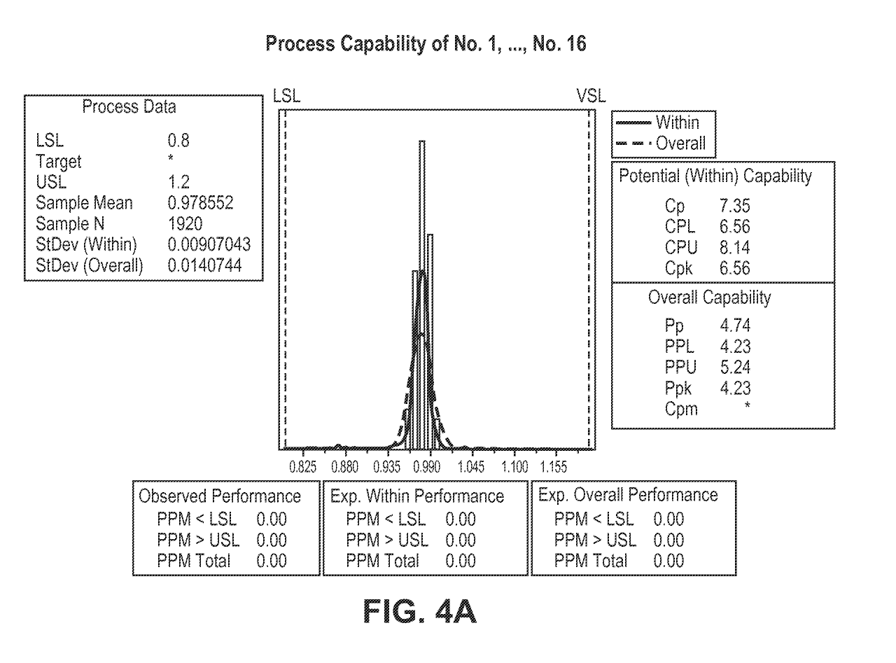

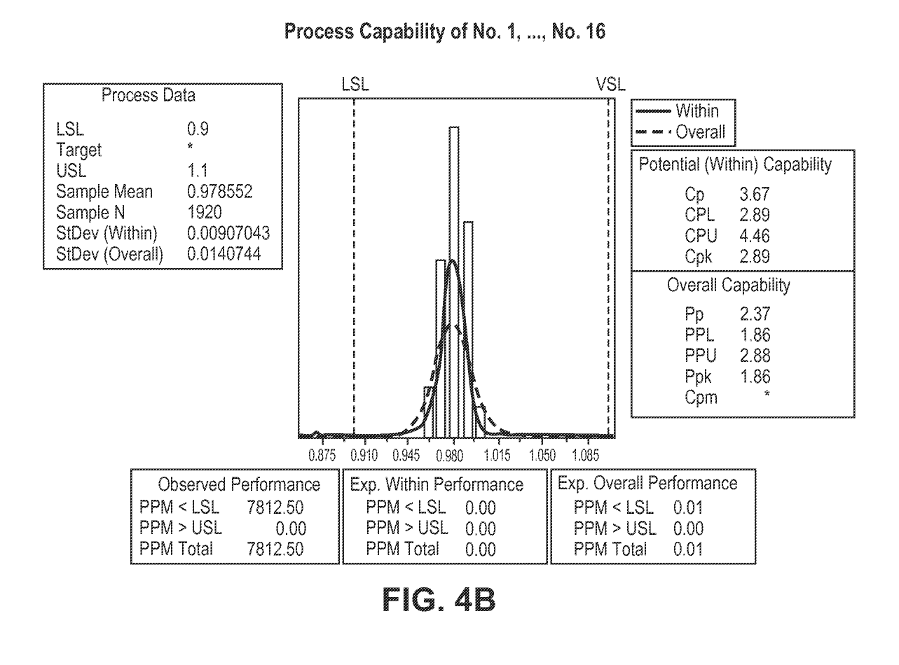

FIGS. 4A-4B summarize the capability analyses results.

Table 2A (below) shows comparative, empirical data among other pump designs that were tested. Three pumps were tested for priming requirements, accuracy and precision of dispensed amounts, residual product left in the bottles, etc. Accuracy and precision are reflected in the table.

TABLE 2A
Comparison Data of Different Pump Designs

| All cells are in weight* | Pump A | | |
|---|---|---|---|
| (g) | (embodiment) | Pump B | Pump C |
| Individual Container - | 0.99 | 1.04 | 1.23 |
| Maximum Mean | | | |
| Individual Container - | 0.97 | 1.02 | 1.12 |
| Minimum Mean | | | |
| All Containers -Mean | 0.98 | 1.03 | 1.18 |
| All Containers - | 0.01 | 0.03 | 0.06 |
| Standard Deviation | | | |
| Maximum Single | 1.00 | 1.09 | 1.32 |
| Actuation | | | |
| Minimum Single | 0.93** | 0.77 | 0.51 |
| Actuation | | | |

*Calculations were based on the first 120 consecutive doses after priming sessions.
**Unit dose No. 33 was dispensed on January 3rd, 2011, two weeks after the previous dose was dispensed.

As shown in the table, the design of Pump A dispensed product more accurately (i.e., within 0.02 g of 1.00 g) than Pump B (i.e., within 0.03 g of 1.00 g) and Pump C (i.e., within 0.18 g of 1.00 g). Accuracy in dispensing is important so that a product does not need to be re-formulated (e.g., diluted, made stronger) in order for the pump to dispense the correct dose. Avoiding re-formulation saves time and effort in gaining regulatory acceptance of the product for market. An accurate and precise dispensed dose, in an inexpensive, reliable, and consumer-friendly dispenser, increases safety and effectiveness of the pain reliever.

Also as shown in the table, the design of Pump A dispensed product more precisely (i.e., within a standard deviation of 0.01 g to its mean) than Pump B (i.e., within a standard deviation of 0.03 g to its mean) and Pump C (i.e., within 0.06 g to its mean). Precision in dispensing is important so that doses are repeatable. Similarly, the maximum and minimum single actuations of product should be close to the mean value so as to minimize dose outliers.

Pump A, which is in accordance with an embodiment of the invention, has thus been shown to be superior to Pumps B and C.

4.3.3. Process Control Analysis

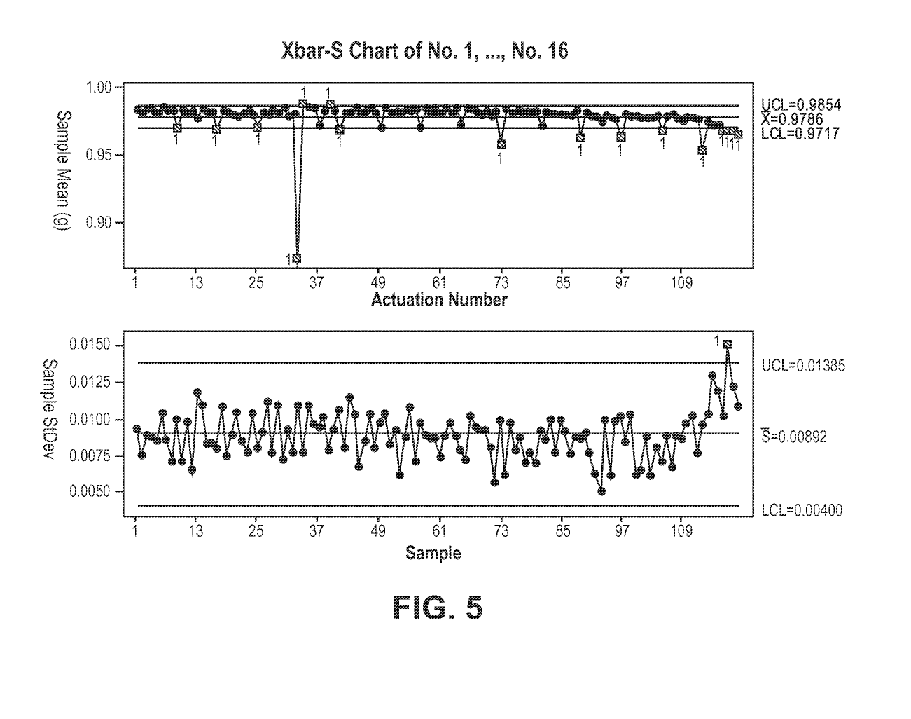

FIG. 5 is an X-bar/S chart that was used to evaluate the statistical control of the dispensing study. Each point in the upper chart represents the mean unit dose weight for all 16 containers, while the lower chart shows all corresponding standard deviations. The graph confirms the overall mean of 0.98 g per unit dose, with a standard deviation of 0.01 g. Although several data points fall outside the ±3σ bands, the overall dispensing appears to be in good statistical control. Dose number 33 is a special cause event due to product evaporation after extended storage. Closer examination of the low doses before the late stage of dispensing showed that many of them coincided with the first unit dose in the morning, again attributed to slight evaporation. Average unit dose weight drop-off at the end is caused by low product quantity in the containers. Overall, unit dose weight remained stable and well within the 0.9-1.1 grange. FIG. 5 shows a statistical process control chart for unit dose weight

4.4. Number of Conforming Doses

All 120 doses analyzed above fall within product specification 0.8-1.2 g, they also meet the following tighter limits proposed in the protocol: within any 20 consecutive doses, no more than 2 doses out of the 0.9-1.1 g and none out of the 0.8-1.2 g range.

Based on above criteria, additional conforming doses were obtained. According to the protocol, three actuations were performed after the first product was discharged. As discussed in 4.2, all three actuations were conforming doses. More conforming doses were also obtained after the dispensing of the 120 unit doses. Overall, the total number of conforming doses ranged from 126 to 132.

4.5. Total Waste

Total waste quantity includes the priming doses, nonconforming doses at the end, and residual left in the container. The average total waste was 6.58 g, with a standard deviation of 1.12 g.

4.6. Inflation Pressure and Fill Capacity

After inflation at 6 psi, all containers were filled to the same level. The average fill weight was 134.06 g, with a standard deviation of 1.21 g. The values varied from 131.57-136.08 g.

Separately from this protocol, a study was conducted to evaluate the impact of inflation pressure on fill capacity. An ANOVA analysis was performed and the results are summarized in below. A comparison of the mean capacity at each pressure was made against capacities at all other pressures, using Fisher's pairwise test. Statistical differences in fill capacities were seen between the following pressures: 3 and 5 & above, 4 and 5 & above, 5 and 7 psi.

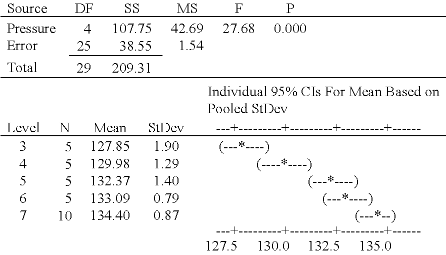

S = 1.242
R-Sq = 81.58%
R-Sq(adj) = 78.63%

The foregoing is an ANOVA summary of the pressure impact on fill capacity.

4.7. Recommended Minimum Fill Weight

The number of conforming doses is directly dependent on container fill weight and unit dose weight. Based on a conservative total waste quantity of 8 g, the following minimum fill weights are suggested at different unit dose weights.

TABLE 3
Recommended minimum fill weights at different unit dose weights

| Mean Unit dose weight | Total conforming dose weight | Minimum fill |
|---|---|---|
| (g) | (g) | weight (g) |
| 0.95 | 114.0 | 122.0 |
| 0.98 | 117.6 | 125.6 |
| 1.00 | 120.0 | 128.0 |
| 1.02 | 122.4 | 130.4 |
| 1.05 | 126.0 | 134.0 |

5. Conclusion

The Rexam Sof'Bag 100 mL container with 1 mL dispensing pump is capable of delivering NLT 120 unit doses of Pennsaid Gel with high precision and accuracy. The mean unit dose weight delivered from all containers is 0.98 g, with a standard deviation of 0.01 g. Dose delivery attributes remained stable over 30 dispensing sessions & one month study period.

6. Deviations

The protocol specified a target fill weight of 132 g (range 131-133 g). However, in order to evaluate fill capacity variability at set pressure of 6 psi, all containers were filled to full capacity. The actual fill weights ranged from 131.57-136.08 g.

### Example 2

### Clinical Pharmacokinetic Analysis of a Topical Viscous Solution of 2.0% w/w Diclofenac Sodium—Multiple Dose

A 2.0% w/w diclofenac sodium topical viscous solution of the present invention was applied to both knees (2 mL [40.4 mg] per knee), topically, every 12±0.5 hours for 7.5 consecutive days in the fed condition. Subjects dispensed the solution from the hand Pump A of Example 1 and applied the topical viscous solution to clean dry skin. To avoid spillage, 2 mL (2 pumps) of the topical viscous solution was dispensed first into the hand and then onto the knee. The topical viscous solution was spread evenly around front, back and sides of knee. The procedure was repeated to the other knee allowing the application to dry completely.

The following pharmacokinetic parameters for diclofenac sodium were determined:
Day 1: The maximum observed plasma concentration (Cₘₐₓ), time to Cₘₐₓ (Tₘₐₓ) and area under plasma concentration curve for the dosing interval 0 to 12 hours (AUC₀₋₁₂) and adjusted to time 0 to 24 hours (AUC₀₋₂₄);
Day 8: Steady-state parameters: AUC₀₋₂₄^{ss}; maximum observed plasma drug concentration at steady-state (Cₘₐₓ^{SS}), observed time to Cₘₐₓ at steady state (Tₘₐₓ^{SS}).

The pharmacokinetic parameters for diclofenac were determined and calculated for the topical viscous solution over the dosing interval of 0 to 12 hours. The arithmetic mean and SD for the pharmacokinetic parameters for diclofenac calculated for the topical viscous solution are presented in the following Table 4:

TABLE 4
Summary of Day 1 and Day 8 Diclofenac Pharmacokinetic Parameters
for Subjects Completing the Study

| | |
|---|---|
| | 2.0% w/w diclofenac |
| | sodium topical V.S. |
| | N = 22 |
| Parameters | Mean (SD) |

Day 1

| | | |
|---|---|---|
| AUC₀₋₁₂ | 95.41 | (80.16) |
| (ng · h/mL) | | |
| AUC₀₋₂₄ | 190.82 | (160.32) |
| (ng · h/mL) | | |
| Cₘₐₓ (ng/mL) | 15.63 | (13.23) |
| Tₘₐₓ (h)^{a} | 12 | (6.00-12.00) |

Day 8

| | | |
|---|---|---|
| AUC₀₋₂₄ | 315.47 | (158.70) |
| (ng · h/mL) | | |
| Cₘₐₓ^{ss} (ng/mL) | 19.53 | (10.31) |
| Tₘₐₓ^{ss} (h)^{a,b} | 1 | (0.00-12.00) |

^{a}Tₘₐₓ and Tₘₐₓ^{SS} are presented as median (range)
^{b}Tₘₐₓ^{SS} is relative to the time of administration of the last dose

After the initial dosing with the topical viscous solution, the mean diclofenac plasma concentrations increased rapidly within 6 hours and continued to rise until 24 hours. Steady-state measurements (before the morning dose on Days 2 through 8) showed that diclofenac concentrations remained elevated at approximately 20 ng/mL from 24 to 168 hours. Measurements immediately after final dosing at 168 hours showed diclofenac concentrations increased by approximately 10 fold with a gradual decline to 10 ng/mL at 192 hours (24 hours after the last dose).

### Example 3

### Clinical Pharmacokinetic Analysis of a Topical Viscous Solution of 2.0% w/w Diclofenac Sodium—Multiple Dose

A 2.0% w/w diclofenac sodium topical viscous solution of the present invention was applied to both knees (2 mL [40.4 mg] per knee), topically, twice a day for 7.5 consecutive days. Total daily dose was approximately 162 mg. The topical viscous solution was supplied in metered-dose pump polypropylene bottles containing 120 mL each. Approximately 1 mL of the topical viscous solution was dispensed per pump. Subjects dispensed the solution from the hand Pump A of Example 1 and applied the topical viscous solution to clean dry skin. To avoid spillage, 2 mL (2 pumps) of the topical viscous solution was dispensed first into the hand and then onto the knee. The topical viscous solution was spread evenly around the front, back, and sides of knee. The procedure was repeated on the other knee, allowing the application to dry completely. Treatment was administered BID (6:00 AM and 6:00 PM). The following PK parameters for diclofenac were determined for both treatments:
Day 1: area under the plasma concentration curve for the first dosing interval 0 to 12 h (AUC₀₋₁₂) and adjusted to time 0 to 24 h (AUC₀₋₂₄), maximum observed plasma concentration (Cₘₐₓ), and time of observed maximum plasma concentration (Tₘₐₓ);
Day 8: AUC for the last dosing interval adjusted to time 0 to 24 h (AUC₀₋₂₄^{SS}), Cₘₐₓ at steady state (Cₘₐₓ^{SS}), Tₘₐₓ at steady state (Tₘₐₓ^{SS}).

The AUC₀₋₁₂ and AUC₀₋₁₂^{SS} were calculated for the topical viscous solution, over the dosing interval of 0 through 12 h on Day 1 and Day 8. So that a comparison between the exposure of each treatment could be assessed on a daily basis, AUC₀₋₂₄ and AUC₀₋₂₄^{SS} were calculated as AUC₀₋₁₂ and AUC₀₋₁₂^{SS}×2 for the 2% w/w diclofenac sodium topical viscous solution.

After the initial dosing with the topical viscous solution, the mean diclofenac plasma concentration increased rapidly within 6 h and remained elevated until reaching a mean (SD) of 14.65 ng/mL (12.38) at 24 h. Steady-state measurements (before the morning dose on Days 2 through 8) showed that mean diclofenac concentrations remained between 12 and 16 ng/mL from 24 to 168 h. On Day 8, the topical viscous solution had an AUC₀₋₂₄^{SS} of 322.57 ng·h/mL (52% coefficient of variation [CV]) and a Cₘₐₓ^{SS} of 19.99 ng/mL (51% CV). On Day 8, the topical viscous solution had an AUC₀₋₂₄^{SS} of 251.24 ng·h/mL (61% CV) and a Cₘₐₓ^{SS} of 14.61 ng/mL (63% CV).

High inter-individual variability is characteristic of topical formulations. The arithmetic mean and SD for the PK parameters for diclofenac calculated for the topical viscous solution are presented in the following Table 5.

TABLE 5
Summary of Day 1 and Day 8 Diclofenac PK Parameters for Subjects
Completing the Trial

| | |
|---|---|
| | 2.0% w/w diclofenac |
| | sodium topical V.S. |
| | N = 29 |
| Parameters | Mean (SD) |

Day 1

| | | |
|---|---|---|
| AUC₀₋₁₂ | 99.54 | (86.48) |
| (ng · h/mL) | | |
| AUC₀₋₂₄ | 199.07 | (172.96) |
| (ng · h/mL) | | |
| Cₘₐₓ (ng/mL) | 15.53 | (12.98) |
| Tₘₐₓ (h)^{a} | 12 | (4.00-12.00) |

Day 8

| | | |
|---|---|---|
| AUC₀₋₂₄ | 322.57 | (167.81) |
| (ng · h/mL) | | |
| Cₘₐₓ^{ss} (ng/mL) | 19.99 | (10.15) |
| Tₘₐₓ^{ss} (h)^{a,b} | 6.5 | (0.00-12.00) |

^{a}Tₘₐₓ and Tₘₐₓ^{SS} are presented as median (range)
^{b}Tₘₐₓ^{SS} is relative to the time of administration of the last dose

### Example 4

Table 6 presents the diclofenac pharmacokinetic parameters of 51 healthy human volunteers topically administered a 2.0% w/w diclofenac sodium topical viscous solution of the present invention (dispensed from the hand Pump A of Example 1) to both knees (2 mL [40.4 mg] per knee), topically, twice a day for 7.5 consecutive days.

TABLE 6

| | |
|---|---|
| | 2.0% w/w diclofenac |
| | sodium topical V.S. |
| Parameters | N = 51 |

Day 1

| | |
|---|---|
| AUC₀₋₂₄ | 195.51 + 166.03 |
| (ng · h/mL) | |
| Cₘₐₓ (ng/mL) | 15.57 + 12.96 |

Day 8

| | |
|---|---|
| AUC₀₋₂₄^{ss} | 319.51 + 162.36 |
| (ng · h/mL) | |
| Cₘₐₓ^{ss} (ng/mL) | 19.79 + 10.12 |

Data represents mean +/− Standard Deviation

### VI. Sample Label

Highlights of Prescribing Information: These highlights do not include all the Information needed to use PENNSAID® PUMP safely and effectively. See full prescribing information for PENNSAID PUMP. PENNSAID PUMP (diclofenac sodium topical solution) 2% w/w is for topical use only. Initial U.S. Approval: 1988.

Warning: Cardiovascular And Gastrointestinal Risk: See full prescribing information for complete boxed warning. Cardiovascular Risk: Nonsteroidal anti-inflammatory drugs (NSAIDs) may cause an increased risk of serious cardiovascular thrombotic events, myocardial infarction, and stroke, which can be fatal. Patients with cardiovascular disease or risk factors for cardiovascular disease may be at greater risk. (5.1) PENNS AID PUMP is contraindicated for the treatment of perioperative pain in the setting of coronary artery bypass graft (CABG) surgery. (4) Gastrointestinal Risk: NSAIDs, including PENNSAID PUMP, cause an increased risk of serious gastrointestinal adverse events including bleeding, ulceration, and perforation of the stomach or intestines, which can be fatal. These events can occur at any time during use and without warning symptoms. Elderly patients are at greater risk for serious gastrointestinal events. (5.2)

Indications and Usage: PENNSAID PUMP is a nonsteroidal anti-inflammatory drug (NSAID) indicated for the treatment of signs and symptoms of osteoarthritis of the knee(s). (1)

Dosage and Administration: For the relief of the signs and symptoms of osteoarthritis of the knee(s): the recommended dose of PENNSAID PUMP is 2 mL on each painful knee, 2 times a day. (2) Apply PENNSAID PUMP to clean, dry skin. (2.1) Dispense 2 pumps of PENNSAID PUMP directly onto the knee or first into the hand and then onto the knee. Spread PENNSAID PUMP evenly around front, back and sides of the knee. (2.1) Wash hand completely after administering the product. (2.2) Wait until the area is completely dry before covering with clothing or applying sunscreen, insect repellent, cosmetics, topical medications, or other substances. (2.2) Until the treated knee(s) is completely dry, avoid skin-to-skin contact between other people and the treated knee(s). (2.2)

Dosage Forms and Strengths: 2% w/w topical solution. (3)

Contraindications: Known hypersensitivity to diclofenac sodium. (4) History of asthma, urticaria, or allergic-type reactions after taking aspirin or other NSAIDs. (4) Use in the perioperative period of coronary artery bypass graft (CABG) surgery. (4)

Warnings and Precautions: Serious and potentially fatal cardiovascular thrombotic events, myocardial infarction, and stroke can occur with NSAID treatment. Use the lowest effective dose of PENNSAID PUMP in patients with known cardiovascular (CV) disease or risk factors for CV disease. (5.1) NSAIDs can cause serious gastrointestinal (GI) adverse events including inflammation, bleeding, ulceration, and perforation. Prescribe PENNSAID PUMP with caution in those with a prior history of ulcer disease or gastrointestinal bleeding. (5.2) Elevation of one or more liver tests may occur during therapy with NSAIDs. Discontinue PENNS AID PUMP immediately if abnormal liver tests persist or worsen. (5.3) Hypertension can occur with NSAID treatment. Monitor blood pressure closely with PENNSAID PUMP treatment. (5.4) Use PENNSAID PUMP with caution in patients with fluid retention or heart failure. (5.5) Long-term administration of NSAIDs can result in renal papillary necrosis and other renal injury. Use PENNSAID PUMP with caution in patients at greatest risk of this reaction, including the elderly, those with impaired renal function, heart failure, liver dysfunction, and those taking diuretics and ACE-inhibitors. (5.6) Anaphylactoid reactions may occur in patients with the aspirin triad or in patients without prior exposure to PENNSAID PUMP. (5.7) NSAIDs can cause serious skin adverse events such as exfoliative dermatitis, Stevens-Johnson Syndrome (SJS), and toxic epidermal necrolysis (TEN), which can be fatal. (5.8) Not for use during pregnancy. (5.9) Do not administer to patients with aspirin sensitive asthma and use with caution in patients with preexisting asthma. (5.10) Avoid exposure of treated knee(s) to natural or artificial sunlight. (5.11) Avoid contact of PENNSAID PUMP with eyes and mucosa. (5.12) Avoid concurrent use with oral NSAIDs. (5.13)

Adverse Reactions: The most common adverse events with PENNSAID PUMP are application site reactions. (6.1) To report suspected adverse reactions, contact Mallinckrodt Brand Pharmaceuticals, Inc. at 1-800-778-7898 or FDA at 1-800-FDA-1088 or www.fda.gov/medwatch.

Drug Interactions: Concomitant administration of diclofenac and aspirin is not generally recommended because of the potential of increased adverse effects including increased GI bleeding. (7.1) Concomitant use of anticoagulants and diclofenac have a risk of serious GI bleeding higher than users of either drug alone. (7.2)

Use in Specific Populations: Pregnancy: Not recommended for use during pregnancy. (8.1) Nursing Mothers: Use with caution, as it is not known if diclofenac is excreted in human milk. (8.3)

See 17 for Patient Counseling Information and Medication Guide.

Full Prescribing Information: Contents*. Warning: Cardiovascular and Gastrointestinal Risk. 1. Indications and Usage; 2. Dosage and Administration, 2.1 General Instructions, 2.2 Special Precautions; 3. Dosage Forms and Strengths; 4. Contraindications; 5. Warnings and Precautions, 5.1 Cardiovascular Thrombotic Events, 5.2 Gastrointestinal Effects—Risk of GI Ulceration, Bleeding, and Perforation, 5.3 Hepatic Effects, 5.4 Hypertension, 5.5 Congestive Heart Failure and Edema, 5.6 Renal Effects, 5.7 Anaphylactoid Reactions, 5.8 Skin Reactions, 5.9 Pregnancy, 5.10 Preexisting Asthma, 5.11 Sun Exposure, 5.12 Eye Exposure, 5.13 Oral Nonsteroidal Anti-Inflammatory Drugs, 5.14 Corticosteroid Treatment, 5.15 Inflammation, 5.16 Hematological Effects, 5.17 Monitoring; 6. Adverse Reactions, 6.1 Clinical Studies Experience, 6.2 Postmarketing Experience; 7. Drug Interactions, 7.1 Aspirin, 7.2 Anticoagulants, 7.3 ACE-Inhibitors, 7.4 Diuretics, 7.5 Lithium, 7.6 Methotrexate, 7.7 Cyclosporine, 7.8 Oral Nonsteroidal Anti-Inflammatory Drugs, 7.9 Topical Treatments; 8. Use in Specific Populations, 8.1 Pregnancy, 8.2 Labor and Delivery, 8.3 Nursing Mothers, 8.4 Pediatric Use, 8.5 Geriatric Use; 10. Overdosage; 11. Description; 12. Clinical Pharmacology, 12.1 Mechanism of Action, 12.2 Pharmacodynamics, 12.3 Pharmacokinetics, 12.4 Platelets; 13. Nonclinical Toxicology, 13.1 Carcinogenesis, Mutagenesis, Impairment of Fertility, 13.2 Animal Toxicology and/or Pharmacology; 14. Clinical Studies, 14.1 Pivotal Studies in Osteoarthritis of the Knee; 16. How Supplied/Storage and Handling; 17. Patient Counseling Information, 17.1 Patient/Caregiver Instructions, 17.2 Cardiovascular Effects, 17.3 Gastrointestinal Effects, 17.4 Hepatotoxicity, 17.5 Adverse Skin Reactions, 17.6 Weight Gain and Edema, 17.7 Anaphylactoid Reactions, 17.8 Effects During Pregnancy, 17.9 Eye Exposure. *Sections or subsections omitted from the full prescribing information are not listed.

Full Prescribing Information. Warning: Cardiovascular and Gastrointestinal Risk Cardiovascular Risk: Nonsteroidal anti-inflammatory drugs (NSAIDs) may cause an increased risk of serious cardiovascular thrombotic events, myocardial infarction, and stroke, which can be fatal. This risk may increase with duration of use. Patients with cardiovascular disease or risk factors for cardiovascular disease may be at greater risk [see Warnings and Precautions (5.1)]. PENNSAID PUMP is contraindicated in the perioperative setting of coronary artery bypass graft (CABG) surgery [see Contraindications (4)]. Gastrointestinal Risk: NSAIDs cause an increased risk of serious gastrointestinal adverse events including bleeding, ulceration, and perforation of the stomach or intestines, which can be fatal. These events can occur at any time during use and without warning symptoms. Elderly patients are at greater risk for serious gastrointestinal events [see Warnings and Precautions (5.2)].

1. Indications and Usage: PENNSAID PUMP is a nonsteroidal anti-inflammatory drug (NSAID) indicated for the treatment of signs and symptoms of osteoarthritis of the knee(s).

2. Dosage and Administration. 2.1 General Instructions: For relief of the signs and symptoms of osteoarthritis (OA) of the knee(s), the recommended dose is 2 mL (2 pumps) per knee, 2 times a day. Patients should be instructed to prime the pump before using it for the first time. Instruct patients to fully depress the pump mechanism (actuation) 4 times while holding the bottle in an upright position. This portion should be discarded to ensure proper priming of the pump. No further priming of the bottle should be required. After the priming procedure, patients should completely depress the pump 2 times to achieve the prescribed dosage for one knee. The product may be delivered directly into the palm of the hand and then applied to the desired knee. Apply PENNSAID PUMP evenly around front, back, and sides of knee. To treat the other knee, if symptomatic, repeat the procedure. Apply PENNSAID PUMP to clean, dry skin. Application of PENNSAID PUMP in an amount exceeding or less than the recommended dose has not been studied and is therefore not recommended.

2.2 Special Precautions: Avoid showering/bathing for at least 30 minutes after the application of PENNSAID PUMP to the treated knee. Wash and dry hands after use. Do not apply PENN SAID PUMP to open wounds. Avoid contact of PENNSAID PUMP with eyes and mucous membranes. Do not apply external heat and/or occlusive dressings to treated knees. Avoid wearing clothing over the PENNSAID PUMP-treated knee(s) until the treated knee is dry. Protect the treated knee(s) from natural and artificial sunlight. Wait until the treated area is dry before applying sunscreen, insect repellant, lotion, moisturizer, cosmetics, or other topical medication to the same knee you have just treated with PENNSAID PUMP. Until the treated knee(s) is completely dry, avoid skin-to-skin contact between other people and the treated knee(s).

3. Dosage Forms and Strengths: 2% w/w topical solution.

4. Contraindications: PENNSAID PUMP is contraindicated in patients with a known hypersensitivity to diclofenac sodium or any other component of PENNSAID PUMP. PENNSAID PUMP is contraindicated in patients who have experienced asthma, urticaria, or allergic-type reactions after taking aspirin or other NSAIDs. Severe, rarely fatal, anaphylactic-like reactions to NSAIDs have been reported in such patients [see Warnings and Precautions (5.7, 5.10)]. PENNSAID PUMP is contraindicated in the setting of coronary artery bypass graft (CABG) surgery [see Warnings and Precautions (5.1)].

5. Warnings and Precautions. 5.1 Cardiovascular Thrombotic Events: Clinical trials of several oral COX-2 selective and nonselective NSAIDs of up to three years duration have shown an increased risk of serious cardiovascular (CV) thrombotic events, myocardial infarction (MI), and stroke, which can be fatal. All NSAIDs, including PENNSAID PUMP and COX-2 selective and nonselective orally administered NSAIDs, may have a similar risk. Patients with known CV disease or risk factors for CV disease may be at greater risk. To minimize the potential risk for an adverse CV event in patients treated with an NSAID, use the lowest effective dose for the shortest duration possible. Physicians and patients should remain alert for the development of such events, even in the absence of previous CV symptoms. Inform patients about the signs and/or symptoms of serious CV events and the steps to take if they occur. Two large, controlled, clinical trials of an orally administered COX-2 selective NSAID for the treatment of pain in the first 10 to 14 days following CABG surgery found an increased incidence of myocardial infarction and stroke [see Contraindications (4)]. There is no consistent evidence that concurrent use of aspirin mitigates the increased risk of serious CV thrombotic events associated with NSAID use. The concurrent use of aspirin and NSAIDs, such as diclofenac, does increase the risk of serious gastrointestinal (GI) events [see Warnings and Precautions (5.2)].

5.2 Gastrointestinal Effects: Risk of GI Ulceration, Bleeding, and Perforation NSAIDs, including diclofenac, can cause serious GI adverse events including bleeding, ulceration, and perforation of the stomach, small intestine, or large intestine, which can be fatal. These serious adverse events can occur at any time, with or without warning symptoms, in patients treated with NSAIDs. Only one in five patients who develop a serious upper GI adverse event on NSAID therapy is symptomatic. Upper GI ulcers, gross bleeding, or perforation caused by NSAIDs occur in approximately 1% of patients treated for 3 to 6 months, and in about 2 to 4% of patients treated for one year. These trends continue with longer duration of use, increasing the likelihood of developing a serious GI event at some time during the course of therapy. However, even short-term therapy is not without risk.

Prescribe NSAIDs, including PENNSAID PUMP, with extreme caution in those with a prior history of ulcer disease or gastrointestinal bleeding. Patients with a prior history of peptic ulcer disease and/or gastrointestinal bleeding who use NSAIDs have a greater than 10-fold increased risk for developing a GI bleed compared to patients with neither of these risk factors. Other factors that increase the risk of GI bleeding in patients treated with NSAIDs include concomitant use of oral corticosteroids or anticoagulants, longer duration of NSAID therapy, smoking, use of alcohol, older age, and poor general health status. Most spontaneous reports of fatal GI events are in elderly or debilitated patients and therefore, use special care when treating this population. To minimize the potential risk for an adverse GI event, use the lowest effective dose for the shortest possible duration. Remain alert for signs and symptoms of GI ulceration and bleeding during diclofenac therapy and promptly initiate additional evaluation and treatment if a serious GI adverse event is suspected. For high-risk patients, consider alternate therapies that do not involve NSAIDs.

5.3 Hepatic Effects: Borderline elevations (less than 3 times the upper limit of the normal [ULN] range) or greater elevations of transaminases occurred in about 15% of oral diclofenac-treated patients in clinical trials of indications other than acute pain. Of the markers of hepatic function, ALT (SGPT) is recommended for the monitoring of liver injury. In clinical trials of an oral diclofenac-misoprostol combination product, meaningful elevations (i.e., more than 3 times the ULN) of AST (SGOT) occurred in about 2% of approximately 5,700 patients at some time during diclofenac treatment (ALT was not measured in all studies). In an open-label, controlled trial of 3,700 patients treated for 2 to 6 months, patients with oral diclofenac were monitored first at 8 weeks and 1,200 patients were monitored again at 24 weeks. Meaningful elevations of ALT and/or AST occurred in about 4% of the 3,700 patients and included marked elevations (>8 times the ULN) in about 1% of the 3,700 patients. In this open-label study, a higher incidence of borderline (less than 3 times the ULN), moderate (3 to 8 times the ULN), and marked (>8 times the ULN) elevations of ALT or AST was observed in patients receiving diclofenac when compared to other NSAIDs. Elevations in transaminases were seen more frequently in patients with osteoarthritis than in those with rheumatoid arthritis. Almost all meaningful elevations in transaminases were detected before patients became symptomatic. Abnormal tests occurred during the first 2 months of therapy with oral diclofenac in 42 of the 51 patients in all trials who developed marked transaminase elevations. In postmarketing reports, cases of drug-induced hepatotoxicity have been reported in the first month, and in some cases, the first 2 months of NSAID therapy.

Postmarketing surveillance has reported cases of severe hepatic reactions, including liver necrosis, jaundice, fulminant hepatitis with and without jaundice, and liver failure. Some of these reported cases resulted in fatalities or liver transplantation. In a European retrospective population-based, case-controlled study, 10 cases of oral diclofenac associated drug-induced liver injury with current use compared with non-use of diclofenac were associated with a statistically significant 4-fold adjusted odds ratio of liver injury. In this particular study, based on an overall number of 10 cases of liver injury associated with diclofenac, the adjusted odds ratio increased further with female gender, doses of 150 mg or more, and duration of use for more than 90 days.

Measure transaminases (ALT and AST) periodically in patients receiving long-term therapy with diclofenac, because severe hepatotoxicity may develop without a prodrome of distinguishing symptoms. The optimum times for making the first and subsequent transaminase measurements are not known. Based on clinical trial data and postmarketing experiences, monitor transaminases within 4 to 8 weeks after initiating treatment with diclofenac. However, severe hepatic reactions can occur at any time during treatment with diclofenac. If abnormal liver tests persist or worsen, if clinical signs and/or symptoms consistent with liver disease develop, or if systemic manifestations occur (e.g., eosinophilia, rash, abdominal pain, diarrhea, dark urine, etc.), discontinue PENNSAID PUMP immediately. To minimize the possibility that hepatic injury will become severe between transaminase measurements, inform patients of the warning signs and symptoms of hepatotoxicity (e.g., nausea, fatigue, lethargy, diarrhea, pruritus, jaundice, right upper quadrant tenderness, and “flu-like” symptoms), and the appropriate action to take if these signs and symptoms appear.

To minimize the potential risk for an adverse liver-related event in patients treated with PENNSAID PUMP, use the lowest effective dose for the shortest duration possible. Exercise caution when prescribing PENNSAID PUMP with concomitant drugs that are known to be potentially hepatotoxic (e.g., acetaminophen, certain antibiotics, antiepileptics). Caution patients to avoid taking unprescribed acetaminophen while using PENNSAID PUMP.

5.4 Hypertension: NSAIDs, including diclofenac, can lead to new onset or worsening of preexisting hypertension, either of which may contribute to the increased incidence of CV events. Use NSAIDs, including PENNSAID PUMP, with caution in patients with hypertension. Monitor blood pressure (BP) closely during the initiation of NSAID treatment and throughout the course of therapy. Patients taking ACE-inhibitors, thiazides or loop diuretics may have impaired response to these therapies when taking NSAIDs.

5.5 Congestive Heart Failure and Edema: Fluid retention and edema have been observed in some patients treated with NSAIDs, including PENNSAID PUMP. Use PENNSAID PUMP with caution in patients with fluid retention or heart failure.

5.6 Renal Effects: Use caution when initiating treatment with PENNSAID PUMP in patients with considerable dehydration. Long-term administration of NSAIDs has resulted in renal papillary necrosis and other renal injury. Renal toxicity has also been seen in patients in whom renal prostaglandins have a compensatory role in the maintenance of renal perfusion. In these patients, administration of an NSAID may cause a dose-dependent reduction in prostaglandin formation and, secondarily, in renal blood flow, which may precipitate overt renal decompensation. Patients at greatest risk of this reaction are those with impaired renal function, heart failure, liver dysfunction, those taking diuretics and ACE-inhibitors, and the elderly. Discontinuation of NSAID therapy is usually followed by recovery to the pretreatment state.

No information is available from controlled clinical studies regarding the use of PENNSAID PUMP in patients with advanced renal disease. Therefore, treatment with PENNSAID PUMP is not recommended in patients with advanced renal disease. If PENNSAID PUMP therapy is initiated, close monitoring of the patient's renal function is advisable.

5.7 Anaphylactoid Reactions: As with other NSAIDs, anaphylactoid reactions may occur in patients without prior exposure to PENNSAID PUMP. Do not prescribe PENNSAID PUMP to patients with the aspirin triad. This symptom complex typically occurs in asthmatic patients who experience rhinitis with or without nasal polyps, or who exhibit severe, potentially fatal bronchospasm after taking aspirin or other NSAIDs [see Contraindications (4) and Warnings and Precautions (5.10)]. Seek emergency help in cases where an anaphylactoid reaction occurs.

5.8 Skin Reactions: Do not apply PENNSAID PUMP to open skin wounds, infections, inflammations, or exfoliative dermatitis, as it may affect absorption and tolerability of the drug. NSAIDs, including PENNSAID PUMP, can cause serious skin adverse events such as exfoliative dermatitis, Stevens-Johnson syndrome (SJS), and toxic epidermal necrolysis (TEN), which can be fatal. These serious events may occur without warning. Inform patients about the signs and symptoms of serious skin manifestations, and discontinue use of the drug at the first appearance of skin rash or any other signs of hypersensitivity.

5.9 Pregnancy: PENNSAID PUMP should not be used by pregnant or nursing women or those intending to become pregnant.

5.10 Preexisting Asthma: Patients with asthma may have aspirin-sensitive asthma. The use of aspirin in patients with aspirin-sensitive asthma has been associated with severe bronchospasm, which can be fatal. Since cross-reactivity, including bronchospasm, between aspirin and other nonsteroidal anti-inflammatory drugs has been reported in such aspirin-sensitive patients, do not administer PENNSAID PUMP to patients with this form of aspirin sensitivity and use with caution in patients with preexisting asthma.

5.11 Sun Exposure: Instruct patients to avoid exposure to natural or artificial sunlight on treated knee(s) because studies in animals indicated topical diclofenac treatment resulted in an earlier onset of ultraviolet light-induced skin tumors. The potential effects of PENNSAID PUMP on skin response to ultraviolet damage in humans are not known.

5.12 Eye Exposure: Avoid contact of PENNSAID PUMP with eyes and mucosa. Advise patients that if eye contact occurs, immediately wash out the eye with water or saline and consult a physician if irritation persists for more than an hour.

5.13 Oral Nonsteroidal Anti-Inflammatory Drugs: Concomitant use of oral NSAIDs with PENNSAID PUMP resulted in a higher rate of rectal hemorrhage, more frequent abnormal creatinine, urea and hemoglobin. Therefore, do not use combination therapy with PENNSAID PUMP and an oral NSAID unless the benefit outweighs the risk and conduct periodic laboratory evaluations.

5.14 Corticosteroid Treatment: PENNSAID PUMP cannot be expected to substitute for corticosteroids or to treat corticosteroid insufficiency. Abrupt discontinuation of corticosteroids may lead to exacerbation of corticosteroid-response illness. For patients on prolonged corticosteroid therapy, taper slowly if a decision is made to discontinue corticosteroids.

5.15 Inflammation: The pharmacological activity of PENNSAID PUMP in reducing inflammation, and possibly fever, may diminish the utility of these diagnostic signs in detecting complications of presumed noninfectious, painful conditions.

5.16 Hematological Effects: The effects of PENNSAID 1.5% on platelet function were studied in 10 healthy subjects administered 80 drops four times a day for 7 days. There was no significant change in platelet aggregation following one week of treatment [see Clinical Pharmacology (12.4)]. Anemia is sometimes seen in patients receiving NSAIDs. This may be due to fluid retention, occult or gross GI blood loss, or an incompletely described effect upon erythropoiesis. Check hemoglobin or hematocrit of patients on PENNSAID PUMP if they exhibit any signs or symptoms of anemia or blood loss. NSAIDs inhibit platelet aggregation and have been shown to prolong bleeding time in some patients. Unlike aspirin, their effect on platelet function is quantitatively less, of shorter duration and reversible. Carefully monitor patients receiving PENNSAID PUMP who may be adversely affected by alterations in platelet function, such as those with coagulation disorders or patients receiving anticoagulants.

5.17 Monitoring: Because serious GI tract ulcerations and bleeding can occur without warning symptoms in patients taking NSAIDs, monitor patients for signs or symptoms of GI bleeding. Check CBC and a chemistry profile periodically in patients on long-term treatment with NSAIDs. Discontinue PENNSAID PUMP if abnormal liver tests or renal tests persist or worsen.

6. Adverse Reactions. 6.1 Clinical Studies Experience: Because clinical trials are conducted under widely varying conditions, adverse reaction rates observed in the clinical trials of a drug cannot be directly compared to rates in the clinical trials of another drug and may not reflect the rates observed in practice.

PENNSAID 1.5%: The data described below reflect exposure to PENNSAID 1.5% of 911 patients treated between 4 and 12 weeks (mean duration of 49 days) in seven Phase 3 controlled trials, as well as exposure of 793 patients treated in an open-label study, including 463 patients treated for at least 6 months, and 144 patients treated for at least 12 months. The population mean age was approximately 60 years, 89% of patients were Caucasians, 64% were females, and all patients had primary osteoarthritis. The most common adverse events with PENNSAID 1.5% were application site skin reactions. These events were the most common reason for withdrawing from the studies.

Application site reactions: In controlled trials, application site reactions were characterized by one or more of the following: dryness, erythema, induration, vesicles, paresthesia, pruritus, vasodilation, acne, and urticaria. The most frequent of these reactions were dry skin (32%), contact dermatitis characterized by skin erythema and induration (9%), contact dermatitis with vesicles (2%) and pruritus (4%). In one controlled trial, a higher rate of contact dermatitis with vesicles (4%) was observed after treatment of 152 subjects with the combination of PENN SAID 1.5% and oral diclofenac. In the open label uncontrolled long-term safety study, contact dermatitis occurred in 13% and contact dermatitis with vesicles in 10% of patients, generally within the first 6 months of exposure, leading to a withdrawal rate for an application site event of 14%.

Adverse events common to the NSAID class: In controlled trials, subjects treated with PENNSAID 1.5% experienced some adverse events associated with the NSAID class more frequently than subjects using placebo (constipation, diarrhea, dyspepsia, nausea, flatulence, abdominal pain, edema; see Table 1). The combination of PENNSAID 1.5% and oral diclofenac, compared to oral diclofenac alone, resulted in a higher rate of rectal hemorrhage (3% vs. less than 1%), and more frequent abnormal creatinine (12% vs. 7%), urea (20% vs. 12%), and hemoglobin (13% vs. 9%), but no difference in elevation of liver transaminases.

Table 7 lists all adverse reactions occurring in >1% of patients receiving PENNSAID 1.5%, where the rate in the PENNSAID 1.5% group exceeded placebo, from seven controlled studies conducted in patients with osteoarthritis. Since these trials were of different durations, these percentages do not capture cumulative rates of occurrence.

TABLE 7
Adverse Reactions Occurring in ≧1% of Patients Treated with
PENNSAID 1.5% Topical Solution in Placebo and Oral
Diclofenac-Controlled Trials

| | Treatment Group: | | | |
|---|---|---|---|---|
| | PENNSAID 1.5% | | Topical Placebo | |
| | N = 911 | | N = 332 | |
| Adverse Reaction* | N (%) | | N (%) | |
| Dry Skin (Application Site) | 292 | (32) | 17 | (5) |
| Contact Dermatitis | 83 | (9) | 6 | (2) |
| (Application Site) | | | | |
| Dyspepsia | 72 | (8) | 13 | (4) |
| Abdominal Pain | 54 | (6) | 10 | (3) |
| Flatulence | 35 | (4) | 1 | (<1) |
| Pruritis (Application Site) | 34 | (4) | 7 | (2) |
| Diarrhea | 33 | (4) | 7 | (2) |
| Nausea | 33 | (4) | 3 | (1) |
| Pharyngitis | 40 | (4) | 13 | (4) |
| Constipation | 29 | (3) | 1 | (<1) |
| Edema | 26 | (3) | 0 | |
| Rash (Non-Application Site) | 25 | (3) | 5 | (2) |
| Infection | 25 | (3) | 8 | (2) |
| Ecchymosis | 19 | (2) | 1 | (<1) |
| Dry Skin (Non-Application Site) | 19 | (2) | 1 | (<1) |
| Contact Dermatitis, vesicles | 18 | (2) | 0 | |
| (Application Site) | | | | |
| Paresthesia (Non-Application Site) | 14 | (2) | 3 | (<1) |
| Accidental Injury | 22 | (2) | 7 | (2) |
| Pruritus (Non-Application Site) | 15 | (2) | 2 | (<1) |
| Sinusitis | 10 | (1) | 2 | (<1) |
| Halitosis | 11 | (1) | 1 | (<1) |
| Application Site Reaction | 11 | (1) | 3 | (<1) |
| (not otherwise specified) | | | | |

*Preferred Term according to COSTART

PENNSAID PUMP: The data described below reflect exposure to PENNSAID PUMP of 130 patients treated for 4 weeks (mean duration of 28 days) in one Phase 2 controlled trial. This population's mean age was approximately 60 years, 85% of patients were White, 65% were females, and all patients had primary osteoarthritis. The most common adverse events with PENNSAID PUMP were application site skin reactions. These events were the most common reason for withdrawing from the studies.

Application site reactions: In this controlled trial, application site reactions were characterized by one or more of the following: dryness (22%), exfoliation (7%), erythema (4%), pruritus (2%), pain (2%), induration (2%), rash (2%), and scabbing (<1%). Study subjects were allowed to use emollients to the administration site only after a skin reaction had occurred and were limited to applying these only once per day, in the evening at least 30 minutes following the application of the study drug. Generally, the incidence of these treatment-emergent adverse events (TEAEs) was comparable between the PENNSAID PUMP topical solution group and the vehicle control group, however, the incidence of application site erythema and application site pruritus was notably lower in the PENNSAID PUMP 2% group compared with the vehicle control group.

Adverse events common to the NSAID class: Table 8 lists all adverse reactions occurring in >1% of patients receiving PENNSAID PUMP, where the rate in the PENNSAID PUMP group exceeded vehicle, from a controlled study conducted in patients with osteoarthritis.

TABLE 8
Incidence of TEAEs Occurring in >1% of Subjects with Osteoarthritis
Using PENNSAID PUMP Topical Solution and More Often than in
Subjects with OA Using Vehicle Control (Pooled)

| | PENNSAID | |
|---|---|---|
| | 2% | Vehicle Control |
| | N = 130 | N = 129 |
| MedDRA* Preferred Term | n (%) | n (%) |
| Urinary tract infection | 4 (3%) | 1 (<1%) |
| Application site induration | 2 (2%) | 1 (<1%) |
| Contusion | 2 (2%) | 1 (<1%) |
| Sinus congestion | 2 (2%) | 1 (<1%) |
| Nausea | 2 (2%) | 0 |

*MeDRA = Medical Dictionary for Regulatory Activities

6.2 Postmarketing Experience: In postmarketing surveillance, the following adverse reactions have been reported during post-approval use of PENNSAID 1.5%. Because these reactions are reported voluntarily from a population of uncertain size, it is not always possible to reliably estimate their frequency or establish a causal relationship to drug exposure. Body as a Whole: abdominal pain, accidental injury, allergic reaction, asthenia, back pain, body odor, chest pain, edema, face edema, halitosis, headache, lack of drug effect, neck rigidity, pain. Cardiovascular: palpitation, cardiovascular disorder. Gastrointestinal: diarrhea, dry mouth, dyspepsia, gastroenteritis, decreased appetite, lip swelling, mouth ulceration, nausea, rectal hemorrhage, ulcerative stomatitis, swollen tongue. Metabolic and Nutritional: creatinine increased. Musculoskeletal: leg cramps, myalgia. Nervous: depression, dizziness, drowsiness, lethargy, paresthesia, paresthesia at application site. Respiratory: asthma, dyspnea, laryngismus, laryngitis, pharyngitis, throat swelling. Skin and Appendages: At the Application Site: contact dermatitis, contact dermatitis with vesicles, dry skin, pruritus, rash, skin burning sensation; Other Skin and Appendages Adverse Reactions: eczema, rash, pruritus, skin discoloration, urticarial. Special Senses: abnormal vision, blurred vision, cataract, ear pain, eye disorder, eye pain, taste perversion. Vascular: blood pressure increased, hypertension.

Drug Interactions: Drug interactions with the use of PENNSAID PUMP have not been studied. The following drug interactions [Sections 7.1 to 7.7] are noted for oral diclofenac sodium.

7.1 Aspirin: When diclofenac is administered with aspirin, the binding of diclofenac to protein is reduced, although the clearance of free diclofenac is not altered. The clinical significance of this interaction is not known; however, as with other NSAIDs, concomitant administration of diclofenac and aspirin is not generally recommended because of the potential of increased adverse effects.

7.2 Anticoagulants: The effects of anticoagulants such as warfarin and NSAIDs on GI bleeding are synergistic, such that users of both drugs together have a risk of serious GI bleeding higher than users of either drug alone.

7.3 ACE-Inhibitors: NSAIDs may diminish the antihypertensive effect of angiotensin converting enzyme (ACE) inhibitors. Consider this interaction in patients taking NSAIDs concomitantly with ACE-inhibitors.

7.4 Diuretics: Clinical studies, as well as postmarketing observations, have shown that NSAIDs can reduce the natriuretic effect of furosemide and thiazides in some patients. The response has been attributed to inhibition of renal prostaglandin synthesis. During concomitant therapy with NSAIDs, observe the patient closely for signs of renal failure [see Warnings and Precautions (5.6)], as well as to assure diuretic efficacy.

7.5 Lithium: NSAIDs have produced an elevation of plasma lithium levels and a reduction in renal lithium clearance. The mean minimum lithium concentration increased 15% and the renal clearance was decreased by approximately 20%. These effects have been attributed to inhibition of renal prostaglandin synthesis by the NSAID. Thus, when NSAIDs, including diclofenac, and lithium are administered concurrently, observe patients carefully for signs of lithium toxicity.

7.6 Methotrexate: NSAIDs have been reported to competitively inhibit methotrexate accumulation in rabbit kidney slices. This may indicate that they could enhance the toxicity of methotrexate. Use caution when NSAIDs, including diclofenac, are administered concomitantly with methotrexate.

7.7 Cyclosporine: Diclofenac, like other NSAIDs, may affect renal prostaglandins and increase the toxicity of certain drugs. Therefore, concomitant therapy with diclofenac may increase cyclosporine's nephrotoxicity. Use caution when diclofenac is administered concomitantly with cyclosporine.

7.8 Oral Nonsteroidal Anti-Inflammatory Drugs: Concomitant use of oral NSAIDs with PENNSAID 1.5% has been evaluated in one Phase 3 controlled trial and in combination with oral diclofenac, compared to oral diclofenac alone, resulted in a higher rate of rectal hemorrhage (3% vs. less than 1%), and more frequent abnormal creatinine (12% vs. 7%), urea (20% vs. 12%) and hemoglobin (13% vs. 9%). Therefore, do not use combination therapy with PENNSAID PUMP and an oral NSAID unless the benefit outweighs the risk and conduct periodic laboratory evaluations.

7.9 Topical Treatments: Instruct patients that before applying sunscreen, insect repellant, lotion, moisturizer, cosmetics, or other topical medication to the same skin surface of the knee treated with PENNSAID PUMP, they must wait until the treated area is completely dry.

8. Use in Specific Populations: 8.1 Pregnancy: Pregnancy Category C prior to 30 weeks gestation; Category D starting 30 weeks gestation.

Teratogenic Effects: There are no adequate and well-controlled studies of PENNSAID PUMP in pregnant women. PENNSAID PUMP should not be used by pregnant women as its safe use has not been adequately determined and starting at 30 weeks gestation, diclofenac and other NSAIDs should be avoided by pregnant women as premature closure of the ductus arteriosus in the fetus may occur. Developmental studies in animals demonstrated that diclofenac sodium administration did not produce teratogenicity despite the induction of maternal toxicity and fetal toxicity in mice at doses up to 20 mg/kg/day (0.6-fold the maximum recommended human dose [MRHD] of 154 mg/day based on body surface area comparison), and in rats and rabbits at doses up to 10 mg/kg/day (approximately 0.6-fold and 1.3-fold the MRHD, respectively). Published reproductive and developmental studies of dimethyl sulfoxide (DMSO, the solvent used in PENNSAID PUMP) are equivocal as to potential teratogenicity.

Nonteratogenic Effects: In rats, maternally toxic doses of diclofenac were associated with dystocia, prolonged gestation, reduced fetal weights and growth, and reduced fetal survival.

8.2 Labor and Delivery: The effects of PENNSAID PUMP on labor and delivery in pregnant women are unknown. In rat studies maternal exposure to diclofenac, as with other NSAID drugs, known to inhibit prostaglandin synthesis, increased the incidence of dystocia, delayed parturition, and decreased offspring survival.

8.3 Nursing Mothers: It is not known whether this drug is excreted in human milk; however, there is a case report in the literature indicating that diclofenac can be detected at low levels in breast milk. Because many drugs are excreted in human milk and because of the potential• for serious adverse reactions in nursing infants from PENNSAID PUMP, a decision should be made whether to discontinue nursing or to discontinue the drug, taking into account the importance of the drug to the mother.

8.4 Pediatric Use: Safety and effectiveness in pediatric patients have not been established.

8.5 Geriatric Use: Of the 911 patients treated with PENNSAID 1.5% in seven controlled Phase 3 clinical trials, 444 subjects were 65 years of age and over. There was no age-related difference in the incidence of adverse events. Of the 793 patients treated with PENNSAID 1.5% in one open-labeled safety trial, 334 subjects were 65 years of age and over including 107 subjects 75 and over. There was no difference in the incidence of adverse events with long-term exposure to PENNSAID 1.5% for this elderly population. As with any NSAID, use caution in treating the elderly (65 years and older) and it may be useful to monitor renal function since they are more likely to have decreased baseline renal function.

10. Overdosage: There have been no known experiences of overdose with PENNSAID PUMP. Symptoms following acute NSAID overdose are usually limited to lethargy, drowsiness, nausea, vomiting, and epigastric pain, which are generally reversible with supportive care. Gastrointestinal bleeding can occur. Hypertension, acute renal failure, respiratory depression and coma may occur, but are rare. Anaphylactoid reactions have been reported with therapeutic ingestion of NSAIDs, and may occur following an overdose. Manage patients using symptomatic and supportive care following an NSAID overdose. There are no specific antidotes. Emesis is not recommended due to a possibility of aspiration and subsequent respiratory irritation by DMSO contained in PENNSAID PUMP. Activated charcoal (60 to 100 g in adults, 1 to 2 g/kg in children) and/or osmotic cathartic may be indicated in patients seen within 4 hours of ingestion with symptoms or following a large overdose (5 to 10 times the usual dose). Forced diuresis, alkalinization of urine, hemodialysis, or hemoperfusion may not be useful due to high protein binding. For additional information about overdose treatment, call a poison control center (1-800-222-1222).

11. Description: PENNSAID PUMP is a clear, colorless to faintly pink or orange solution for topical application. PENNSAID PUMP contains 2% w/w diclofenac sodium, a benzeneacetic acid derivative that is a nonsteroidal anti-inflammatory drug, designated chemically as 2-((2,6-dichlorophenyl)aminol-benzeneacetic acid, monosodium salt. The molecular weight is 318.14. Its molecular formula is Cl4H10Cl2NNaO2 and it has the following structural formula:

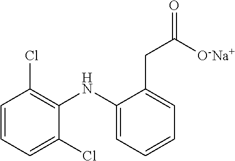

Each 1 mL of solution contains 20.2 mg of diclofenac sodium. In addition, PENNSAID PUMP contains the following inactive ingredients: dimethyl sulfoxide USP (DMSO, 45.5% w/w), ethanol 95% (v/v), purified water, propylene glycol, and hydroxypropyl cellulose.

12. Clinical Pharmacology: 12.1 Mechanism of Action: The mechanism of action of diclofenac is similar to that of other nonsteroidal anti-inflammatory drugs. Diclofenac inhibits the enzyme, cyclooxygenase (COX), an early component of the arachidonic acid cascade, resulting in the reduced formation of prostaglandins, thromboxanes and prostacylin. It is not completely understood how reduced synthesis of these compounds results in therapeutic efficacy.

12.2 Pharmacodynamics: Diclofenac, the active component of PENNSAID PUMP has anti-inflammatory, anti-nociception, and antipyretic effects.

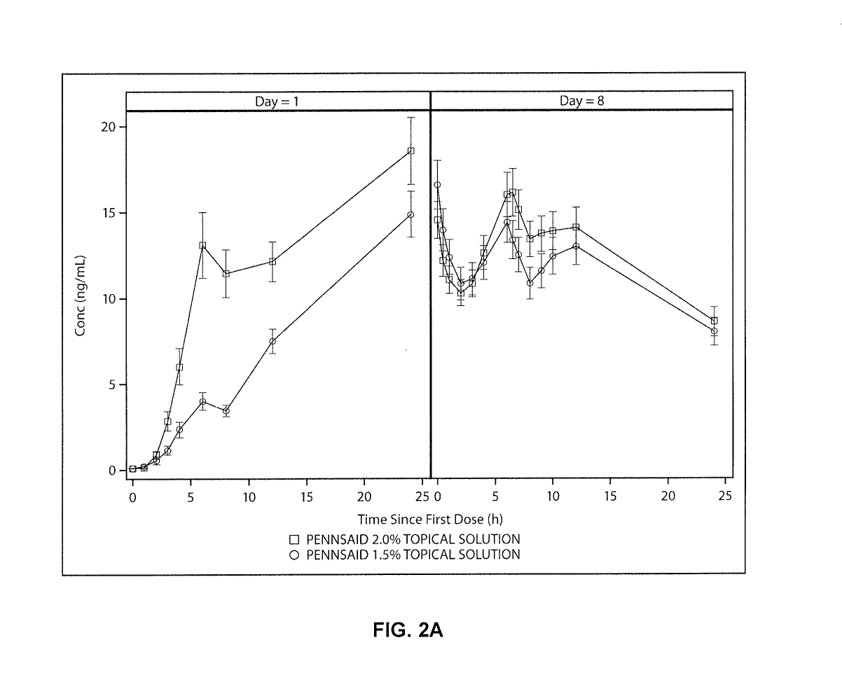

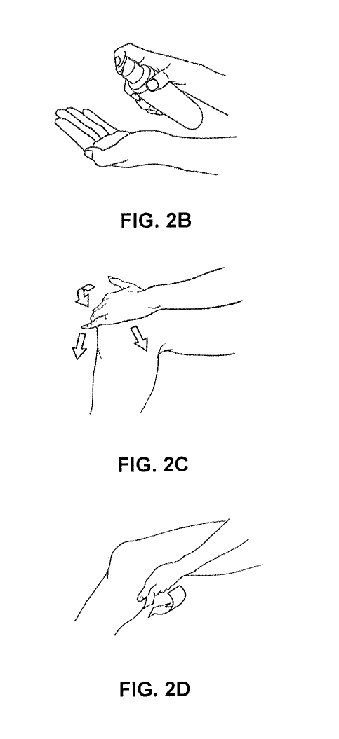

12.3 Pharmacokinetics: After topical administration of PENNSAID 1.5% (40 drops per knee, 80 drops total dose, four times daily) and PENNSAID PUMP (2 mL per knee, 4 mL total dose, twice daily) to healthy human volunteers for 7.5 days, the following diclofenac pharmacokinetic parameters were obtained (see Table 9 and FIG. 2A):

TABLE 9
PENNSAID 1.5% and PENNSAID PUMP Topical Solution
Pharmacokinetic Parameters for Diclofenac Sodium.

| | PENNSAID 1.5% | PENNSAID PUMP |
|---|---|---|
| | Normal Adults | Normal Adults |
| | [N = 51] (Age: 18-55 | [N = 51] (Age: 18-55 |
| Pharmacokinetic | Four times daily (QID) | Two times daily (BID) |
| Parameters | for 7.5 days | for 7.5 days |
| | Day 1 | |
| AUC₀₋₂₄ (ng · h/mL) | 37.52 ± 42.26 | 195.51 ± 166.03 |
| Cₘₐₓ (ng/mL) | 4.14 ± 3.83 | 15.57 ± 12.96 |
| | Day 8 | |
| AUC₀₋₂₄^{SS} (ng · h/mL) | 295.47 ± 168.91 | 319.51 ± 162.36 |
| Cₘₐₓ^{SS} (ng/mL) | 16.10 ± 9.21 | 19.79 ± 10.12 |
| t_{1/2} (h) | 32.53 ± 11.38 | 29.72 ± 10.04 |
| Kₑₗ (h⁻¹) | 0.0236 ± 0.0071 | 0.0264 ± 0.0104 |

Data represents mean +/1 Standard Deviation (SD)

FIG. 2A: Mean+/−Standard Error Diclofenac Concentrations (ng/mL)—Day 1 and Day 8 Completers. FIG. 2A shows the mean diclofenac concentrations with standard error bars after administration of PENNSAID 1.5% and PENNSAID PUMP in healthy volunteers (n=51). The left panel represents Day 1 (0 to 24 hrs) and the right panel represents Day 8 after 7.5 days of administration (0 to 24 hrs).

Absorption: Diclofenac systemic exposure from PENNSAID 1.5% application (4 times daily for 1 week) was approximately ⅓ of the diclofenac systemic exposure from the Solaraze (diclofenac topical gel) application (twice daily for 4 weeks).

Distribution: Diclofenac is more than 99% bound to human serum proteins, primarily to albumin Diclofenac diffuses into and out of the synovial fluid. Diffusion into the joint occurs when plasma levels are higher than those in the synovial fluid, after which the process reverses and synovial fluid levels are higher than plasma levels. It is not known whether diffusion into the joint plays a role in the effectiveness of diclofenac. After repeated dermal application of PENNSAID PUMP to porcine knees, diclofenac concentrations were higher in the skin, synovial fluid and subtended tissues, including muscle, tendon and bone, compared to the plasma.

Metabolism: Five diclofenac metabolites have been identified in human plasma and urine. The metabolites include 4′-hydroxy-, 5-hydroxy-, 3′-hydroxy-, 4′,5-dihydroxy- and 3′-hydroxy-4′-methoxy diclofenac. The major diclofenac metabolite, 4′-hydroxy-diclofenac, has very weak pharmacologic activity. The formation of 4′-hydroxy diclofenac is primarily mediated by CYP2C9. Both diclofenac and its oxidative metabolites undergo glucuronidation or sulfation followed by biliary excretion. Acylglucuronidation mediated by UGT2B7 and oxidation mediated by CYP2C8 may also play a role in diclofenac metabolism. CYP3A4 is responsible for the formation of minor metabolites, 5-hydroxy and 3′-hydroxy-diclofenac.

Excretion: Diclofenac is eliminated through metabolism and subsequent urinary and biliary excretion of the glucuronide and the sulfate conjugates of the metabolites. Little or no free unchanged diclofenac is excreted in the urine.

Special Populations: Pediatric: The pharmacokinetics of PENNSAID PUMP has not been investigated in pediatric patients. Race: Pharmacokinetic differences due to race have not been studied.

12.4 Platelets: The effect of PENNSAID 1.5% on platelet function was evaluated in 10 healthy human volunteers as a sub-study of a multiple-dose pharmacokinetic study [see Pharmacokinetics (12.3)]. Average (range) platelet aggregation time following stimulation with adenosine diphosphate, collagen, epinephrine and arachidonic acid was 101.3% (73.3 to 128.1), 99.8% (69.6 to 112.9), 109.9% (66.2 to 178.1) and 99.0% (15.5 to 126.6) of baseline value, respectively. These results indicate that there was no effect on platelet aggregation after application of the maximum clinical dose for 7 days [see Pharmacokinetics (12.3)].

13. Nonclinical Toxicology: 13.1 Carcinogenesis, Mutagenesis, Impairment of Fertility: Carcinogenicity studies in mice and rats administered diclofenac sodium as a dietary constituent for 2 years resulted in no significant increases in tumor incidence at doses up to 2 mg/kg/day corresponding to approximately 0.35- and 0.7-fold (mouse and rat, respectively) of the maximum recommended human topical dose of PENNSAID PUMP (based on apparent bioavailability and body surface area comparison). In a dermal carcinogenicity study conducted in albino mice, daily topical applications of diclofenac sodium for two years at concentrations up to 0.035% diclofenac sodium (a 43-fold lower diclofenac sodium concentration than present in PENNSAID PUMP) did not increase neoplasm incidence. In a photococarcinogenicity study conducted in hairless mice, topical application of diclofenac sodium at doses up to 0.035% diclofenac sodium (a 43-fold lower diclofenac sodium concentration than present in PENNSAID PUMP) resulted in an earlier median time of onset of tumors.

Mutagenesis: Diclofenac was not mutagenic or clastogenic in a battery of genotoxicity tests that included the bacterial reverse mutation assay, in vitro mouse lymphoma point mutation assay, chromosomal aberration studies in Chinese hamster ovarian cells in vitro, and in vivo rat chromosomal aberration assay of bone marrow cells.

Impairment of Fertility: Fertility studies have not been conducted with PENNSAID PUMP. Diclofenac sodium administered to male and female rats at doses up to 4 mg/kg/day (1.4-fold of the MRHD of PENNSAID PUMP based on apparent bioavailability and body surface area comparison) did not affect fertility. Studies conducted in rats found no effect of dermally applied DMSO on fertility, reproductive performance, or offspring performance.

13.2 Animal Toxicology and/or Pharmacology: Ocular Effects: No adverse effects were observed using indirect ophthalmoscopy after multiple-daily dermal application to rats for 26 weeks and minipigs for 52 weeks of DMSO at twice the concentration found in PENNSAID 1.5%. Published studies of dermal or oral administration of DMSO to rabbits, dogs and pigs described refractive changes of lens curvature and cortical fibers indicative of myopic changes and/or incidences of lens opacity or discoloration when evaluated using slit-lamp biomicroscopy examination, although no ocular abnormalities were observed in rhesus monkeys during daily oral or dermal treatment with DMSO for 9 to 18 months.

14. Clinical Studies: 14.1 Studies in Osteoarthritis of the Knee.

PENNSAID PUMP: The use of PENNSAID PUMP topical solution for the treatment of the signs and symptoms of osteoarthritis of the knee was evaluated in a single double-blind controlled trial conducted in the US, involving patients treated with PENNSAID PUMP at a dose of 2 pumps twice a day for 4 weeks. PENNSAID PUMP was compared to topical vehicle, applied directly to the study knee. In this trial, PENNSAID PUMP treatment resulted in clinical improvement compared to vehicle in all three primary efficacy variables-pain, physical function (Western Ontario and McMaster Universities LK3.1 OA Index (WOMAC) pain and physical function subscales and Patient Global Assessment (PGA). Numerical results are summarized in Table 10.

TABLE 10
Change in Treatment Outcomes after 4 Weeks of Treatment with
PENNSAID PUMP Topical Solution (LOCF/BOCF)*

Treatment

| | PENNSAID | Vehicle |
|---|---|---|
| | PUMP | Control |
| Efficacy Variable | N = 130 | N = 129 |
| WOMAC Pain Subscale | | |
| Baseline | 12.4 | 12.6 |
| Mean Change from Baseline | −4.5 | −3.6 |
| WOMAC Physical Function Subscale | | |
| Baseline | 42.9 | 43.3 |
| Mean Change from Baseline | −14.3 | −11.5 |
| Patient Global Assessment | | |
| Baseline | 4.0 | 4.0 |
| Mean Change from Baseline | −1.1 | −0.9 |

*LOCF—Last observation carried forward/BOCF—Baseline observation carried forward

PENNSAID 1.5%: The use of PENNSAID 1.5% topical solution for the treatment of the signs and symptoms of osteoarthritis of the knee was previously evaluated in two double-blind controlled trials conducted in the US and Canada, involving patients treated with PENNSAID 1.5% at a dose of 40 drops four times a day for 12 weeks. PENN SAID 1.5% was compared to topical placebo (2.3% DMSO with other excipients) and/or topical vehicle solution (45.5% w/w DMSO with other excipients), applied directly to the study knee. In both trials, PENNSAID 1.5% treatment resulted in statistically significant clinical improvement compared to placebo and/or vehicle, in all three primary efficacy variables-pain, physical function (Western Ontario and McMaster Universities LK3.1 OA Index (WOMAC) pain and physical function subscales) and Patient Overall Health Assessment (POHA)/Patient Global Assessment (PGA). Numerical results are summarized in Tables 11 and 12.

TABLE 11
Change in Treatment Outcomes after 12 Weeks of Treatment
in One Study of Efficacy of PENNSAID 1.5%

| | Study I | | | |
|---|---|---|---|---|
| | Mean baseline score and mean | | | |
| | change in efficacy variables | | | |
| | after 12 weeks of treatment | | | |
| | Mean | PENNSAID | Topical | Topical |
| | Baseline | 1.5% | placebo* | vehicle^{†} |
| Efficacy Variable | score | N = 154 | N = 155 | N = 161 |
| WOMAC pain score | 13 | −6.0 | −4.7 | −4.7 |
| (Likert 3.1, 0-20) | | | | |
| WOMAC physical | 42 | −15.7 | −12.3 | −12.1 |
| function | | | | |
| (Likert 3.1, 0-68) | | | | |
| POHA | 2.3 | −1.0 | −0.4 | −0.6 |
| (0-4) | | | | |

*placebo formulation included 2.3% DMSO
^{†}vehicle formulation included 45.5% DMSO

TABLE 12
Change in Treatment Outcomes after 12 Weeks of Treatment
in One Study of Efficacy of PENNSAID 1.5%

| | Study II | | |
|---|---|---|---|
| | Mean baseline score and | | |
| | mean change in efficacy variables | | |
| | after 12 weeks of treatment | | |
| | | PENNSAID | Topical |
| | Mean Baseline | 1.5% | vehicle* |
| Efficacy Variable | score | N = 164 | N = 162 |
| WOMAC pain score | 13 | −5.9 | −4.4 |
| (Likert 3.1, 0-20) | | | |
| WOMAC | 42 | −15.3 | −10.3 |
| physical function | | | |
| (Likert 3.1, 0-68) | | | |
| PGA (0-4) | 3.1 | −1.3 | −1.0 |

*vehicle formulation included 45.5% DMSO

16. How Supplied/Storage and Handling. PENNSAID PUMP is supplied as a clear, colorless to faintly pink or orange solution containing 20.2 mg of diclofenac sodium per mL of solution, in a white polypropylene metered dose pump bottle with a clear cap. NDC Number & Size 112 mL bottle, NDC #23635-510-12. Storage: Store at 25° C. (77° F.); excursions permitted to 15° to 30° C. (59° to 86° F.) [see USP Controlled Room Temperature].

17. Patient Counseling Information. See FDA-approved patient labeling (Medication Guide and Instructions for Use).

17.1 Patient/Caregiver Instructions. Inform patients of the following information before initiating therapy with an NSAID and periodically during the course of ongoing therapy. Encourage patients to read the NSAID Medication Guide that accompanies each prescription dispensed prior to using PENNSAID PUMP [see Medication Guide and Patient Instructions for Use].

17.2 Cardiovascular Effects. PENNSAID PUMP, like other NSAIDs, m a y cause serious CV side effects, such as MI or stroke, which may result in hospitalization and even death. Although serious CV events can occur without warning symptoms, instruct patients to be alert for the signs and symptoms of chest pain, shortness of breath, weakness, slurring of speech, and to ask for medical advice when observing any indicative sign or symptoms. Inform patients of the importance of this follow-up [see Warnings and Precautions (5.1)].

17.3 Gastrointestinal Effects. PENNSAID PUMP, like other NSAIDs, may cause GI discomfort and, rarely, serious GI side effects, such as ulcers and bleeding, which may result in hospitalization and even death. Although serious GI tract ulcerations and bleeding can occur without warning symptoms, inform patients to be alert for the signs and symptoms of ulceration and bleeding, and to ask for medical advice when observing any indicative sign or symptoms including epigastric pain, dyspepsia, melena, and hematemesis. Instruct patients of the importance of this follow-up [see Warnings and Precautions (5.2)].

17.4 Hepatotoxicity. Inform patients of the warning signs and symptoms of hepatotoxicity (e.g., nausea, fatigue, lethargy, pruritus, jaundice, right upper quadrant tenderness, and “flu-like” symptoms). If these occur, instruct patients to stop therapy with PENNSAID PUMP and seek immediate medical therapy [see Warnings and Precautions (5.3)].

17.5 Adverse Skin Reactions. PENNSAID PUMP, like other NSAIDs, can cause serious systemic skin side effects such as exfoliative dermatitis, SJS, and TEN, which may result in hospitalizations and even death. Although serious systemic skin reactions may occur without warning, instruct patients to be alert for the signs and symptoms of skin rash and blisters, fever, or other signs of hypersensitivity such as itching, and to ask for medical advice when observing any indicative signs or symptoms [see Warnings and Precautions (5.8)]. Advise patients to stop PENNSAID PUMP immediately if they develop any type of generalized rash and contact their physicians as soon as possible. PENNSAID PUMP can cause a localized skin reaction at the application site. Advice patients to contact their physicians as soon as possible if they develop any type of localized application site rash. Instruct patients not to apply PENNSAID PUMP to open skin wounds, infections, inflammations, or exfoliative dermatitis, as it may affect absorption and reduce tolerability of the drug. Instruct patients to wait until the area treated with PENNSAID PUMP is completely dry before applying sunscreen, insect repellant, lotion, moisturizer, cosmetics, or other topical medication. Instruct patients to avoid skin-to-skin contact between other people and the knee(s) to which PENNSAID PUMP was applied until the knee(s) is completely dry. Instruct patients to minimize or avoid exposure of treated knee(s) to natural or artificial sunlight.

17.6 Weight Gain and Edema. Instruct patients to promptly report to their physician signs or symptoms of unexplained weight gain or edema following treatment with PENNSAID PUMP [see Warnings and Precautions (5.5)].

17.7 Anaphylactoid Reactions. Inform patients of the signs of an anaphylactoid reaction (e.g., difficulty breathing, swelling of the face or throat). If these occur, instruct patients to seek immediate emergency help [see Warnings and Precautions (5.7)].

17.8 Effects During Pregnancy. Instruct patients who are pregnant or intending to become pregnant not to use PENNSAID PUMP [see Use in Specific Populations (8.1) and Impairment of Fertility (13.1)].

17.9 Eye Exposure. Instruct patients to avoid contact of PENNSAID PUMP with the eyes and mucosa. Advise patients that if eye contact occurs, immediately wash out the eye with water or saline and consult a physician if irritation persists for more than an hour.

Medication Guide for Non-Steroidal Anti-Inflammatory Drugs (NSAIDs). (See the end of this Medication Guide for a list of prescription NSAID medicines.)

What is the most important information I should know about medicines called Non-Steroidal Anti-Inflammatory Drugs (NSAIDs)? NSAID medicines may increase the chance of a heart attack or stroke that can lead to death. This chance increases: with longer use of NSAID medicines, in people who have heart disease. NSAID medicines should never be used right before or after a heart surgery called a “coronary artery bypass graft (CABG).” NSAID medicines can cause ulcers and bleeding in the stomach and intestines at any time during treatment. Ulcers and bleeding: can happen without warning symptoms, may cause death. The chance of a person getting an ulcer or bleeding increases with: taking medicines called “corticosteroids” and “anticoagulants,” longer use, smoking, drinking alcohol, older age, having poor health. NSAID medicines should only be used: exactly as prescribed, at the lowest dose possible for your treatment, for the shortest time needed.

What are Non-Steroidal Anti-Inflammatory Drugs (NSAIDs)? NSAID medicines are used to treat pain and redness, swelling, and heat (inflammation) from medical conditions such as: different types of arthritis, menstrual cramps and other types of short-term pain.

Who should not take a Non-Steroidal Anti-Inflammatory Drug (NSAID)? Do not take an NSAID medicine: if you had an asthma attack, hives, or other allergic reaction with aspirin or any other NSAID medicine, for pain right before or after heart bypass surgery.

Tell your healthcare provider: about all of your medical conditions; about all of the medicines you take—NSAIDs and some other medicines can interact with each other and cause serious side effects. Keep a list of your medicines to show to your healthcare provider and pharmacist; if you are pregnant—NSAID medicines should not be used by pregnant women late in their pregnancy; if you are breastfeeding—Talk to your doctor.

What are the possible side effects of Non-Steroidal Anti-Inflammatory Drugs (NSAIDs)? Serious side effects include: heart attack, stroke, high blood pressure, heart failure from body swelling (fluid retention), kidney problems including kidney failure, bleeding and ulcers in the stomach and intestine, low red blood cells (anemia), life-threatening skin reactions, life-threatening allergic reactions, liver problems including liver failure, asthma attacks in people who have asthma. Other side effects include: stomach pain, constipation, diarrhea, gas, heartburn, nausea, vomiting, dizziness.

Get emergency help right away if you have any of the following symptoms: shortness of breath or trouble breathing, chest pain, slurred speech, weakness in one part or side of your body, swelling of the face or throat.

Stop your NSAID medicine and call your healthcare provider right away if you have any of the following symptoms: nausea, more tired or weaker than usual, itching, your skin or eyes look yellow, stomach pain, flu-like symptoms, vomit blood, there is blood in your bowel movement or it is black and sticky like tar, unusual weight gain, skin rash or blisters with fever, swelling of the arms and legs, hands and feet. These are not all the side effects with NSAID medicines. Talk to your healthcare provider or pharmacist for more information about NSAID medicines. Call your doctor for medical advice about side effects. You may report side effects to FDA at 1-800-FDA-1088.

Other information about Non-Steroidal Anti-Inflammatory Drugs (NSAIDs): Aspirin is an NSAID medicine but it does not increase the chance of a heart attack. Aspirin can cause bleeding in the brain, stomach, and intestines. Aspirin can also cause ulcers in the stomach and intestines. Some of these NSAID medicines are sold in lower doses without a prescription (over-the-counter). Talk to your healthcare provider before using over-the-counter NSAIDs for more than 10 days. As with all topical NSAIDs, secondary exposure may be a possibility when skin-to-skin contact occurs with the application site before the topical NSAID has dried.

NSAID medicines that need a prescription: Generic Name: Celecoxib, Tradename: Celebrex®; Generic Name: Diclofenac, Tradename: Flector, Cataflam®, Voltaren®, Arthrotec™ (combined with misoprostol), PENNSAID® PUMP; Generic Name: Diflunisal, Tradename: Dolobid®; Generic Name: Etodolac, Tradename: Lodine®, Lodine® XL; Generic Name: Fenoprofen, Tradename: Nalfon®, Nalfon® 200; Generic Name: Flurbirofen, Tradename: Ansaid®; Generic Name: Ibuprofen, Tradename: Motrin®, Tab-Profen®, Vicoprofen®* (combined with hydrocodone), Combunox™ (combined with oxycodone); Generic Name: Indomethacin, Tradename: Indocin®, Indocin® SR, Indo-Lemmon™, Indomethagan™; Generic Name: Ketoprofen, Tradename: Oruvail®; Generic Name: Ketorolac, Tradename: Toradol®; Generic Name: Mefenamic Acid, Tradename: Ponstel®; Generic Name: Meloxicam, Tradename: Mobic®; Generic Name: Nabumetone, Tradename: Relafen®; Generic Name: Naproxen, Tradename: Naprosyn®, Anaprox®, Anaprox® DS, EC-Naproxyn®, Naprelan®, Naprapac® (copackaged with lansoprazole); Generic Name: Oxaprozin, Tradename: Daypro®; Generic Name: Piroxicam, Tradename: Feldene®; Generic Name: Sulindac, Tradename: Clinoril®; Generic Name: Tolmetin, Tradename: Tolectin®, Tolectin DS®, Tolectin® 600. *Vicoprofen contains the same dose of ibuprofen as over-the-counter (OTC) NSAID, and is usually used for less than 10 days to treat pain. The OTC NSAID label warns that long term continuous use may increase the risk of heart attack or stroke.

This Medication Guide has been approved by the U.S. Food and Drug Administration.

Patient Instructions for Use. PENNSAID® PUMP [pen/sed]. (diclofenac sodium topical solution). Your doctor has prescribed PENNSAID PUMP to treat your pain from osteoarthritis in your knee(s) and help you manage your daily activities better.

Before you use PENNSAID PUMP: Apply PENNSAID PUMP exactly as your doctor tells you. Do not apply PENNSAID PUMP anywhere on your body other than where your doctor tells you. Apply PENNSAID PUMP on clean, dry skin that does not have any cuts, infections or rashes. Use PENNSAID PUMP two (2) times a day on your knee(s). Do not get PENNSAID PUMP in your eyes, nose or mouth. Only use PENNSAID PUMP on your skin (topical use). If you get PENNSAID PUMP in your eyes, rinse your eyes right away with water or saline. Call your doctor if your eyes are irritated for more than one hour.

Steps for using PENNSAID PUMP (diclofenac sodium topical solution): Before the first use of PENNSAID PUMP, prime the pump by fully depressing the pump mechanism 4 times while holding the bottle in an upright position. Discard this portion of the product to ensure proper priming After the initial priming, it is not necessary to prime the pump again.

Step 1. Wash your hands with soap before applying PENNSAID PUMP. Step 2. Remove the bottle cap and press the pump head down firmly and fully to dispense PENNSAID PUMP into the palm of the hand. Release the pump head and then press the pump head down firmly and fully a second time. For regular, daily use, the bottle can be held at an angle. FIG. 2B. Dispense 2 pumps of PENNSAID PUMP into hand. Step 3. Apply PENNSAID PUMP evenly around front, back, and sides of the knee. PENNSAID PUMP should be applied without massaging the knee. FIG. 2C. Spread PENNSAID PUMP evenly on the front, and sides of your knee. FIG. 2D. Spread PENNSAID PUMP evenly on the back of your knee. Step 4: Repeat Steps 2 and 3 for the other knee if needed. Step 5: After applying PENNSAID PUMP, wash your hands with soap and water. Step 6: Avoid skin-to-skin contact between other people and your knee(s) to which PENNSAID PUMP was applied until your knee(s) is completely dry. Replace the cap on the bottle and store in an upright position.

After you use PENNSAID PUMP: Do not: cover your knee with clothing until your knee is completely dry; put sunscreen, insect repellant, lotion, moisturizer, cosmetics, or other topical medicines on your knee until it is completely dry; take a shower or a bath for at least minutes after you put PENNSAID PUMP on your knee(s); use heating pads or apply bandages to the skin where you have applied PENNSAID PUMP; expose your skin to sunlight or artificial light (tanning booths) where you have put PENNSAID PUMP.

How should I store PENNSAID PUMP? Store PENNSAID PUMP between 59° F. to 86° F. (15° C. to 30° C.). Keep PENNSAID PUMP and all medicines out of the reach of children.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for treating pain of osteoarthritis of the knee of a patient in need thereof, the method comprising:
applying a therapeutically effective amount of a solution of diclofenac sodium to the knee of said patient,
wherein said solution has a viscosity of between 500-3000 centipoise,
wherein said applying comprises dispensing said solution from a pharmaceutically acceptable hand pump and spreading the solution onto the skin of said knee, and
wherein said application provides
a mean Cₘₐₓ of diclofenac of about 10.0 ng/mL to about 25.0 ng/mL;
a mean Cₘₐₓ of diclofenac at steady state of about 12.0 ng/mL to about 30.0 ng/mL; and/or
a mean AUC₀₋₁₂ of diclofenac of about 60.0 nghr/mL to about 140.0 nghr/mL.

2. The method of claim 1, wherein said application provides
a mean Cₘₐₓ of diclofenac of about 10.0 ng/mL to about 25.0 ng/mL;
a mean Cₘₐₓ of diclofenac at steady state of about 12.0 ng/mL to about 30.0 ng/mL; and
a mean AUC₀₋₁₂ of diclofenac of about 60.0 nghr/mL to about 140.0 nghr/mL.

3. The method of claim 1, wherein said application provides
a mean AUC₀₋₁₂ of diclofenac of about 76.0 nghr/mL to about 124.0 nghr/mL.

4. The method of claim 1, wherein said application provides
a mean Cₘₐₓ of diclofenac of about 12.4 ng/mL to about 19.5 ng/mL;

5. The method of claim 1, wherein said viscous solution comprises:
(i) about 2% w/w diclofenac sodium;
(ii) about 40-50% w/w DMSO;
(iii) about 23-29% w/w ethanol;
(iv) about 10-12% w/w propylene glycol;
(v) about 1-10% w/w thickener; and
(vi) water to make 100% w/w.

6. The method of claim 1, wherein said viscous solution consists of:
(i) about 2% w/w diclofenac sodium;
(ii) about 40-50% w/w DMSO;
(iii) about 23-29% w/w ethanol;
(iv) about 10-12% w/w propylene glycol;
(v) about 1-10% w/w thickener; and
(vi) water to make 100% w/w.
